(19) United States
(12) Patent Application Publication
(10) Pub. No.: US 2012/0053446 A1
(43) Pub. Date: Mar. 1, 2012

(54) VOTING IN IMAGE PROCESSING

(75) Inventors: Ilia Lossev, Boulder, CO (US); Natasha Bagotskaya, Boulder, CO (US); Alexander Filatov, Longmont, CO (US); Vadim Nikitin, Boulder, CO (US)

(73) Assignee: Parascript LLC, Longmont, CO (US)

(21) Appl. No.: 13/287,799

(22) Filed: Nov. 2, 2011

Related U.S. Application Data
(63) Continuation-in-part of application No. 12/765,514, filed on Apr. 22, 2010.
Application No. 12/765,514 is a continuation-in-part of application No. 11/943,957, filed on Nov. 21, 2007.

(51) Int. Cl. A61B 6/00; G06K 9/00

(52) U.S. Cl. 600/407; 382/128

(57) ABSTRACT

Methods and systems are to facilitate the interaction between a user and a computer aided detection (“CAD”) system when identifying areas of interest in an image. The CAD system receives input from a user indicating areas of interest that the user perceives in the image. The input may include location information as well as additional information about the area of interest. The CAD system may then perform a voting process using the user provided areas of interest and one or more sets of areas of interest identified by at least one image recognition process to produce a final set of areas of interest in the image. The voting process is useful in eliminating false positives and false negatives that may have been identified by the user and/or the image recognition processes. Further embodiments disclosed herein provide methods and systems for training a voting process to complement a particular user.

### CROSS-REFERENCE TO RELATED CASES

This patent application claims priority to, and is a continuation-in-part of, U.S. patent application Ser. No. 12/765,514 filed on Apr. 22, 2010, entitled “VOTING IN MAMMOGRAPHY PROCESSING,” which is a continuation-in-part of, U.S. patent application Ser. No. 11/943,957 filed on Nov. 21, 2007, entitled “VOTING IN MAMMOGRAPHY PROCESSING,” both of which applications are hereby incorporated by reference in their entirety.

### BACKGROUND

Medical imaging has been utilized in the medical industry for various purposes from detecting broken or fractured bones to identifying the early development of cancer. Medical images are generally analyzed by experts such as radiologists or physicians in order to determine whether the image displays an indication that the patient requires medical treatment. However, many radiologists and physicians analyze hundreds of medical images a day leading to fatigue which may result in human error. Computer applications may be used to mitigate the chance of human error. It is with respect to this general environment that embodiments of the present invention have been contemplated.

### SUMMARY

Embodiments of the present disclosure relate to detecting areas of interest on an image. In embodiments, a user identifies areas of interest in an image and inputs the user identified areas of interest into a device, such as a computer aided detection (“CAD”) device. The device receives the user identified areas of interest along with areas of interest identified by one or more image recognition processes. The device then performs one or more voting processes on the user result set and the one or more result sets from the one or more image recognition processes to produce a final set of areas of interest. The one or more voting processes reduce the number of false positives and false negatives identified by the user and the one or more image recognition processes.

Methods to train a voting process for a particular user are also disclosed. In embodiments, a user's performance related to identifying areas of interest may be compared to known areas of interest in an image or confirmed image data in order to adjust one or more voting processes for a particular user. Adjusting the voting processes for the user allow the systems disclosed herein to apply one or more voting processes that compensate for a user's strengths and weaknesses in identifying areas of interest. This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Embodiments of the present invention may be more readily described by reference to the accompanying drawings in which like numbers refer to like items and in which:

FIG. 1 is an illustration of a mammogram image 100 displaying identified areas of interest.

FIG. 2 is a flow chart representing an embodiment of a method 200 for determining a confidence value for and locations of areas of interest on an image.

FIG. 3 is an illustration of a mammogram image 300 displaying a situation where the boundaries of different identified areas of interest intersect.

FIG. 4 is a flow chart representing an embodiment of a method 400 for applying a voting process based upon an intersection of boundaries of identified areas of interest.

FIG. 5 is an illustration of a mammogram image 500 displaying a situation where sections of two different identified areas of interest overlap.

FIG. 6 is a flow chart representing an embodiment of a method 600 for applying a voting process based upon an overlap of different identified areas of interest.

FIG. 7 is an illustration of a mammogram image 700 displaying a situation where the focal points of different identified areas of interest are compared.

FIG. 8 is a flow chart representing an embodiment of a method 800 for applying a voting process based upon the comparison of focal points of different identified areas of interest.

FIG. 9 is a functional diagram illustrating a computer environment and computer system 900 operable to execute embodiments of the present disclosure.

FIG. 10 is a flow chart representing an embodiment of a method 1000 for applying a voting function to a result set from an image recognition process.

FIG. 11 is a flow chart representing an embodiment of a method 11 for applying voting functions to multiple result sets from multiple image recognition processes.

FIG. 12 is an illustration 1200 of initial areas of interest identified by a first image recognition process on a mammogram image.

FIG. 13 is an illustration 1300 of initial areas of interest identified by a second image recognition process on a mammogram image.

FIG. 14 is an example illustration 1400 of a continuous representation of a first initial area of interest.

FIG. 15 is an example illustration 1500 of a continuous representation of a second initial area of interest.

FIG. 16 is an example illustration 1600 of combined representation for the first image recognition process.

FIG. 17 is an example illustration 1700 of combined representation for the first image recognition process.

FIG. 18 is an embodiment of an illustration 1800 of a unified composite model of the results from first and second image recognition processes.

FIG. 19 is an embodiment of an example output produced by applying voting functions to the results produced by image recognition processes.

FIG. 20 is a flow chart representing a method 2000 for applying a voting process to user provided areas of interest.

FIG. 21 is a flow chart representing a method 2100 for training a voting process for a particular user.

FIG. 22 is a flow chart representing an additional method 2200 for training a voting process for a particular user.

### DETAILED DESCRIPTION

This disclosure will now more fully describe exemplary embodiments with reference to the accompanying drawings, in which some of the possible embodiments are shown. Other aspects, however, may be embodied in many different forms and the inclusion of specific embodiments in the disclosure should not be construed as limiting such aspects to the embodiments set forth herein. Rather, the embodiments depicted in the drawings are included to provide a disclosure that is thorough and complete and which fully conveys the intended scope to those skilled in the art. When referring to the figures, like structures and elements shown throughout are indicated with like reference numerals.

Embodiments of the present disclosure relate to detecting areas of interest in an image. In embodiments, one or more image recognition processes are applied to an image to locate areas of interest on the image. In embodiments, each image recognition process is unique (e.g. each process uses a different algorithm, has a different threshold values, etc.). In one embodiment, the recognition processes do not share the information generated by the process (e.g., information derived from computations, results, etc.). In embodiments, each image recognition process may identify one or more areas of interest on the image. A process may also calculate a confidence value for each area of interest that corresponds to the likelihood that an image recognition process properly identified an area of interest. After identifying the areas of interest, the locations identified by the different algorithms, the resulting areas are compared. In an embodiment, the areas are compared using a voting process. The voting process may calculate a refined confidence value that corresponds to the likelihood that an image recognition process properly identified an area of interest provided that other image recognition processes identified a set of areas of interest that may be accompanied by corresponding confidence values. In further embodiments, the voting process may select specific identified areas of interest calculated by one or more image recognition processes, identify new areas of interest based upon the identified areas of interest calculated by the one or more image recognition processes, or both.

In other embodiments, one or more voting functions are applied to result sets derived from one or more image recognition processes in order to more accurately identify areas of interest. In such embodiments, an area of interest comprises a hypothesis about the significance of a particular portion of image data. The image recognition processes may be designed to identify such areas of interest based on a variety of criteria. Thus, different image recognition processes may produce different result sets identifying different areas of interest. Furthermore, each recognition process may have a different level of confidence attached to their respective results. Applying one or more voting functions effectively combines the different result sets resulting in a final result set that more accurately identifies areas of interest than individual recognition processes on their own.

In embodiments, the resulting areas of interest identify the location of cancer in a mammogram image. In other embodiments, the methods and systems disclosed herein are used to detect lesions, calcifications, tumors, cysts, or other ailments, each of which terms are used interchangeably herein. In embodiments, the areas of interest are identified on the image for further review by a physician. In other embodiments, information about the identified areas of interest is passed to other applications for further processing. While certain methods and systems disclosed herein may be directed towards detecting cancer in mammogram images, one skilled in the art will recognize that the methods and systems may also be practiced on X-ray images, computer axial tomography (“CAT”) scans, magnetic resonance imaging (“MRI's”), or any other type of medical imaging known in the art. In further embodiments, the methods and systems disclosed herein may be applied to images of any organ or tissue to aid in pathology.

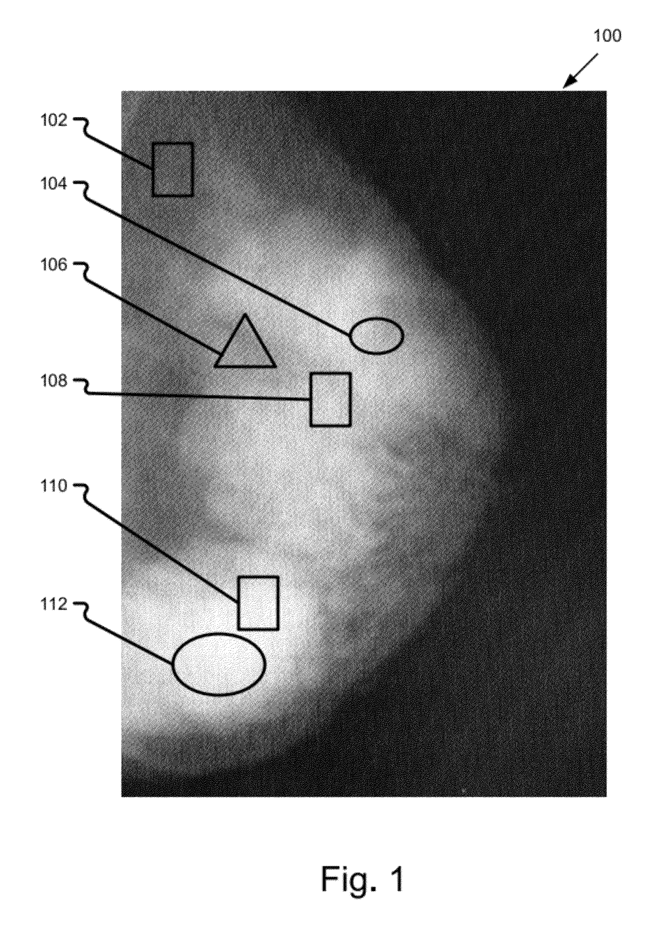

Referring now to FIG. 1, an illustration of a mammogram image 100 displaying identified areas of interest is provided. In embodiments, the methods and systems disclosed herein receive an image, such as mammogram image 100, and apply one or more image identification processes. Image recognition processes may identify areas of interest on an image. Identified areas of interest may be displayed on the image, such as identifications 102, 104, 106, 108, 110, and 112. In embodiments, an image recognition process, such as a rule-based image analyzer or a probabilistic image analyzer, may identify areas of interest on an image by examining specific features of the image, although other image recognition processes may be employed in other embodiments of the present disclosure. Examined features may include image features such as intensity, gradient of intensity, contrast, location, or any other examinable image features known to the art. In other embodiments, image recognition processes may use an algorithm to identify areas of interest on the image (e.g., algorithms using pattern matching, statistical analysis, pattern recognition, etc.) One of skill in the art will appreciate that the disclosed methods and systems will operate regardless of the means employed by the image recognition processes, and that any type of image detection or analysis know to the art may be used.

In embodiments, an image will be processed by at least one image recognition process. In the example illustrated by FIG. 1, three different image recognition processes were used. For example, areas of interest identified by a rectangular border, such as identifications 102, 108, and 110, are areas of interest identified by a first image recognition process. Areas of interest identified by an oval border, such as identifications 104 and 112, may be identified by a second image recognition process, where the second image recognition process is different than the first image recognition process used to identify the areas of interest corresponding to identifications 102, 108, and 110. In embodiments, the areas of interest may be identified independently using different image recognition processes. In such an embodiment, the different image recognition process may not share information used in identifying the areas of interest. In other embodiments, the results of one image recognition process may be input into a second image recognition process. Identification 106, identified by a triangular border may be identified using yet another unique image recognition process. In embodiments, image recognition processes are unique when the processes utilize different means for identifying areas of interest on the image (e.g., examine different features, employ different algorithms, use different thresholds, etc.) Image recognition processes, in embodiments, may output results in the form of identified areas of interest, objects on the image, or nothing (e.g., no identified areas of interest). One of skill in the art will recognize that as long as there is at least one unique image recognition process, any number of additional image recognition processes can be used with the disclosed systems and methods.

Because image recognition processes are inherently imperfect, not every identified area of interest, e.g., identifications 102, 104, 106, 108, 110, and 112, are actual areas of interest. An identified area of interest that is not an actual area of interest is known as a false positive. False positives may be eliminated by determining a confidence value for each identified area of interest, such as identifications 102, 104, 106, 108, 110, and 112. In embodiments, a confidence value may be determined by comparing the identified areas of interest identified by one image recognition process with areas of interest recognized by a second image recognition process. For example, if one or more unique image recognition processes identify the same area, or overlapping areas, or areas closely located to one another, such as identification 110 and identification 112, as an area of interest on the image, there is a higher likelihood that an actual area of interest exists in that area. Thus, the identifications in this instance may be assigned a higher confidence value. Conversely, if an area of interest identified by one image recognition process is remotely located from other identified areas of interest, such as identification 102, it is more likely that the identified area of interest is a false positive (e.g., not an actual area of interest) because the image recognition process or other image recognition processes did not recognize the area as an area of interest. In this situation, a lower confidence value may be assigned to the identification. In embodiments, confidence values may be assigned to the individual identifications themselves or to areas representing a combination of the individual identifications (e.g., new areas of increased interest).

In other embodiments, the confidence value for an identified area of interest may also be determined or adjusted by comparing areas of interest identified by the same image recognition process. For example, identifications 102, 108, and 110, areas of interest identified by the same imager recognition process, may be compared. As a result of the comparison, confidence values for each of the identified areas of interest may be assigned or adjusted.

In embodiments, the comparisons are made using different areas of interest identified by different image recognition processes to determine the confidence value of areas of interest. This differs from using a single image recognition process to determine an area of interest and then re-analyzing the determined area of interest with different image recognition processes. Instead, in embodiments, the entire relevant portion of an image may be analyzed by different image recognition processes or a single image recognition process. In such an embodiment, the recognition process or processes may identify different areas of interest, or objects, on the image. In embodiments, the areas of interest may be identified independently or jointly using more than one image recognition process. In some instances, an image recognition process will not identify any areas of interest or objects on the image. The results of the image recognition process or processes (e.g., determined areas of interest, identified object, or the lack of identification) may then be compared to one another. As a result, comparing the identified areas, or objects, or lack thereof results in a determination of a confidence value for each object.

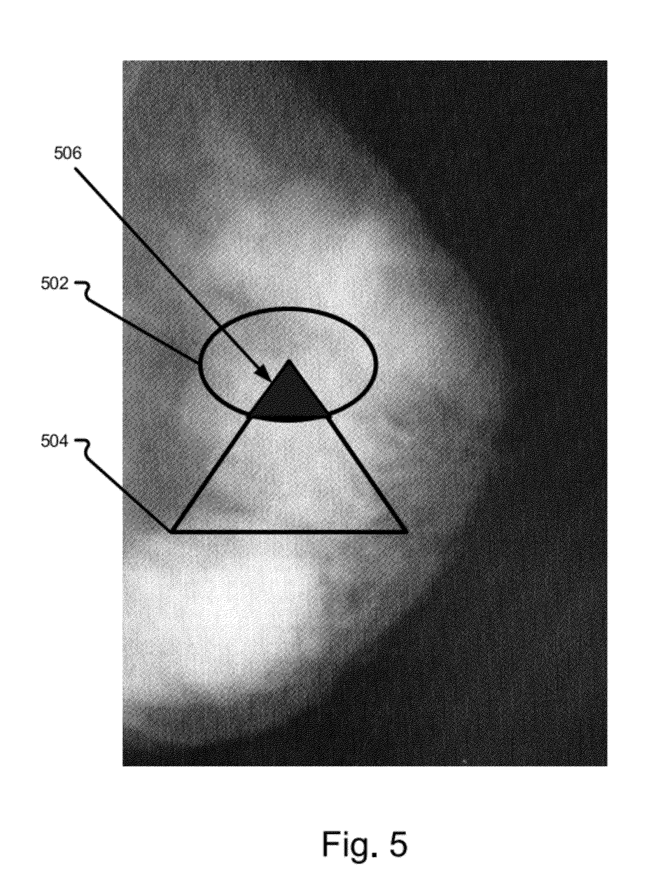

In embodiments, the accuracy of location of identified areas of interest on the image may also be augmented by comparing identified areas of interest identified by at one or more image recognition processes. For example, if two identified areas of interest overlap (See FIG. 5) the comparison may result in a new identified area of interest that contains only the overlapping portion of the two identified areas of interest, as demonstrated by overlapping portion 506 (FIG. 5). Because both areas of interest overlap, there is a higher likelihood that the overlapped area contains an actual area of interest as opposed to the portions of the two identified areas that do not overlap. Thus, the resulting identification identifying the overlapped portion 506 (FIG. 5) of the two identified areas of interest may represent a more accurate identification of an area of interest. In general, a new set of areas of interest may be created based on a set of original areas of interest identified by one or more image recognition processes. The new areas of interest may be determined by selecting the overlapping portions of original identified areas of interest, by combining the original identified areas of interest, or by creating more complex areas of interest based upon the original identified areas of interest. While embodiments of the present figure have been described using specific markings (e.g., oval, rectangle, and triangle boundaries), one of skill in the art will appreciate that any form of image labeling known to the art may be practiced with embodiments of the present disclosure.

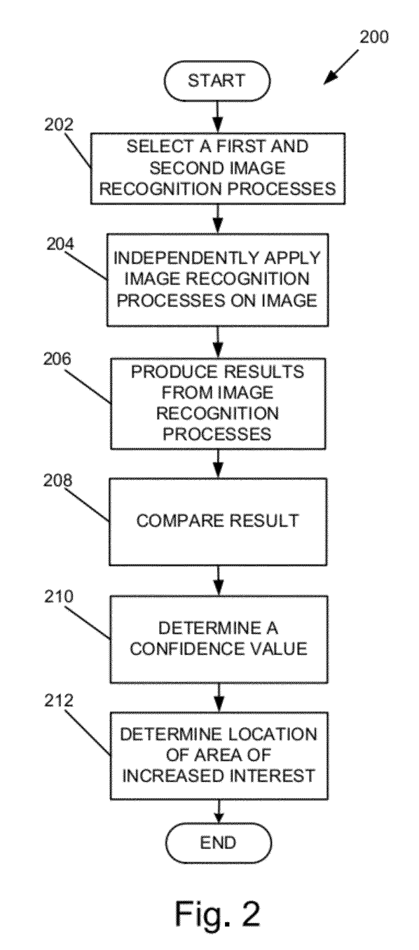

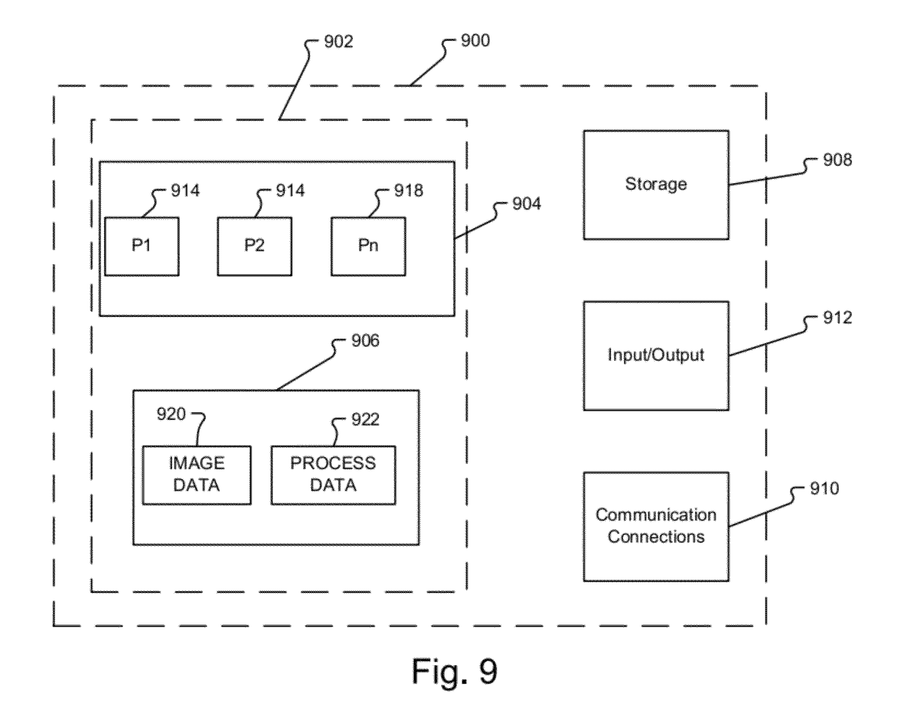

FIG. 2 is a flow chart representing an embodiment of a method 200 for determining a confidence value for and locations of areas of interest on an image. In embodiments, flow begins at select operation 202 where first and second image recognition processes are selected by the method. In embodiments, the selected image recognition processes are unique. In another embodiment, the selected image recognition processes may be the same. Flow then proceeds to apply operation 204 where the selected image recognition processes are applied to an image. In one embodiment, the image recognition processes are applied independently such that no information is shared between the processes, thus allowing the processes to independently identify areas of interest on the image. In another embodiment the image recognition processes may share some information. In one embodiment, the image recognition processes are performed serially. In other embodiments, the image recognition processes are performed in parallel to save computing time. In such embodiments, a parallel processor system may be utilized to provide the computational power necessary to perform parallel image recognition processes. In embodiments, each processor of a parallel processor system is dedicated to each image recognition algorithm in order to spread the workload across multiple processors and increase computational efficiency. Other distributed and parallel computing processes will be recognized by those of skill in the art. Embodiments of computer systems operable to execute embodiments of the disclosure are explained below with regards to FIG. 9.

Flow may then proceed to produce operation 206. Produce operation 206 may produce indications of identified areas of interest produced by each image recognition process on the image. For example, FIG. 1 is an embodiment illustrating indications of various identified areas of interest produced by various image recognition processes. In some embodiments, produce operation 206 is not performed and/or the results of produce operation 206 are not presented to a user, but are used as inputs to compare operation 208.

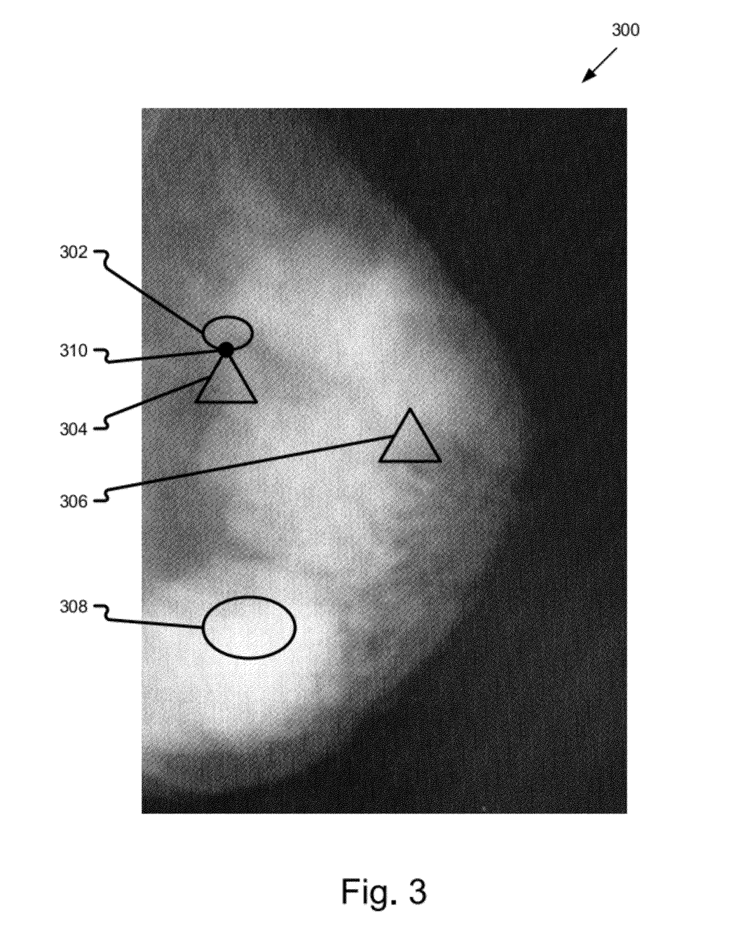

Flow then proceeds to compare operation 208, where the results (e.g., the identified areas of interest or objects) of the first and second image recognition processes are compared. For example, the areas of interest or objects on the image identified by the one or more image recognition processes are compared. In embodiments, the comparison is accomplished using a voting process. Various embodiments of voting processes are further described below with reference to FIGS. 3-8. In embodiments, the results of the compare operation 208 are used in determining a confidence value in confidence operation 210 and/or in determining a location of areas of increased interest in location operation 212. In other embodiments, comparisons and/or voting may be used to produce a confidence value when a first image recognition process outputs an area of interest and a second image recognition process outputs nothing. For example, if a first image recognition process identifies one or more areas of interest on an image and a second image recognition process does not, the confidence values for the identified areas of interest may be adjusted accordingly to take into account the results, or lack thereof, of the second image analyzer.

Flow proceeds to confidence operation 210, where a confidence value is determined for the identified areas of interest. In embodiments, the confidence value is based upon the comparison or comparisons made in comparison operation 210. In embodiments, if an area of interest identified by the first image recognition process is collocated with, overlapping, or located near an area of interest identified by a second image recognition process, the identified areas may be assigned a higher confidence value, as previously described with reference to FIG. 1. Conversely, in embodiments, if an area of interest identified by the first or second image recognition processes is located remotely from other identified areas of interest, then the remote area of interest may be assigned a lower confidence value. Confidence values for new areas of interest (e.g., combined areas of interest identified by one or more image recognition processes, overlapping portions, etc.) may also be determined at operation 210.

In location operation 212, a determination is made as to the locations of identified areas of interest that most likely correspond to actual areas of interest. An actual area of interest is an area on the image that actually displays sought after features (e.g., cancer in a mammogram image). In embodiments, the determination is based upon the confidence value of each identified area of interest assigned in confidence operation 210. In one embodiment, only identified areas of interest meeting a certain threshold related to one or more confidence values are selected. These selected areas are areas of increased interest due to their confidence value meeting a required threshold. The threshold of confidence may be predefined, determined during the operation of method 200, or determined by a user. These areas of increased interest are selected because they are more likely to be actual areas of interest and less likely to be false positives. In this embodiment, only indications of areas of increased interest meeting the threshold of confidence are displayed on the image. In embodiments, indications of areas of increased interest may be displayed on the image by highlighting the areas of increased interest, enclosing the areas of increased interest in a border, marking the areas of increased interest, or by any other method of image labeling known in the art.

In another embodiment, location operation 212 may create new areas of increased interest based upon the comparisons made in compare operation 208. For example, if identified areas of interest overlap, location operation 212 may create new areas of increased interest that correspond only to the overlapping portion of the identified areas of interest. In this embodiment, indications of the new areas of increased interest are displayed on the image. In yet another embodiment, identified areas of interest meeting a threshold of confidence and new areas of increased interest produced at location operation 212 are displayed on the image. Although operations 208, 210, and 212 have been described as independent operations, one of skill in the art will appreciate that these operations may be accomplished in one step (e.g., the compare operation 208 may also assign confidence values and determine locations of areas of increased interest).

Referring now to FIG. 3, an illustration of a mammogram image 300 displaying a situation where the boundaries of different identified areas of interest intersect is provided. FIG. 3 displays areas of interest identified by two image recognition processes at identifications 302, 304, 306, and 308. In other embodiments, indications 302, 304, 306, and 308 may be identified by a single image recognition process or by more than two image recognition processes. A first image process identified two areas of interest, identifications 302 and 308 represented as ovals. A second image process identified an additional two areas of interest identifications 304 and 306 represented as triangles. In embodiments, identifications 302, 304, 306, and 308 may be identified by one or more image recognition processes or the identifications may be identified using a combination of image recognition processes. The borders of identifications 302 and 304 intersect, at intersection point 310. The intersection of the borders indicates a higher probability that an area of increased interest exists in the vicinity of the identified areas of interest 302 and 304 because two different image recognition processes identified the vicinity as an area of interest. In other embodiments, two identified areas of interest in the same vicinity may result in higher confidence values. Therefore, the identified areas of interest represented by identifications 302 and 304 may be assigned a higher confidence. In one embodiment, identified areas of interest represented by identifications 302 and 304 may be assigned a higher confidence. In another embodiment, the areas where identifications 302 and 304 intersect are assigned a higher confidence. In yet another embodiment, identifications 302 and 304 are assigned a higher confidence and a new area of increased interest around the intersection points is also assigned a higher confidence. In this embodiment, the confidence assigned to each of the areas may or may not be the same (e.g., the new area of increased interest may have a higher confidence than identifications 302 and 304). Conversely, indications 306 and 308 are located remotely from all other indications. In embodiments, indications 306 and 308 may be determined to be remote from other indications because their borders do not intersect the borders of other indications. Because these indications are remotely located, there is a higher likelihood that these indications represent false negatives, and therefore may be assigned lower confidence values. FIG. 3 is an embodiment in which the comparison performed in compare operation 208 (FIG. 2) may use a voting process that determines whether the boundaries of areas of interest intersect.

In another embodiment an automatic learning process based, for example, on statistical methods or neural networks is utilized to determine a confidence value for each area based on such features as a confidence value assigned by an image recognition process that identified the area, locations of areas identified by other recognition processes, corresponding confidence values, etc. In embodiments, the areas of interest, or objects, separately identified by different image recognition processes which analyze an entire image are compared to determine confidence levels. While embodiments of the present figure have been described using specific markings (e.g., oval and triangle boundaries), one of skill in the art will appreciate that any form of image labeling known to the art may be practiced with embodiments of the present disclosure. One of skill in the art will also appreciate that while embodiments of the present disclosure have been explained in regards to analyzing mammogram images, any type of image may be analyzed using embodiments of the present disclosure.

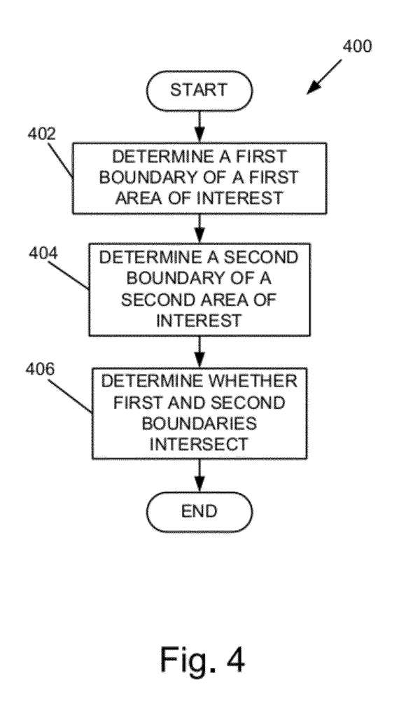

FIG. 4 is a flow chart representing an embodiment of a method 400 for applying a voting process based upon an intersection of boundaries of identified areas of interest. Flow begins at operation 402, where the method determines a boundary for a first area of interest identified by a first image recognition process. In one embodiment, the boundary may be determined by maintaining a list of pixels on the image corresponding to the boundary of the first identified area of interest. In embodiments, the image may be divided into nodes rather than pixels. In this embodiment, the nodes representing the border of the identified area of interest are determined. In another embodiment, the boundary may be determined by defining a mathematical formula representing the boundary of the first identified area of interest. One skilled in the art will appreciate that any method of determining a boundary for an area of an image may be employed at operation 402.

Flow then proceeds to operation 404, where the method determines a boundary for a second area of interest identified by a second image recognition process. In embodiments, the second area of interest is identified by the second image recognition process. In another embodiment, the second area of interest is defined by the same image recognition process that identified the first area of interest. Again, in one embodiment, the boundary may be determined by maintaining a list of pixels on the image corresponding to the boundary of the second identified area of interest. In embodiments, the image may be divided into nodes rather than pixels. In this embodiment, the nodes representing the border of the identified area of interest are determined. In another embodiment, the boundary may be determined by defining a mathematical formula representing the boundary of the second identified area of interest. One skilled in the art will appreciate that any method of determining a boundary for an area of an image may be employed at operation 404.

Once the boundaries for both identified areas of interest have been determined, flow proceeds to operation 406, where the method computes the intersection of the first and second boundaries. In one embodiment, the determination may be made by comparing the pixels representing the first boundary to the pixels representing the second boundary. If the same pixel is present in both boundaries, the borders intersect. In another embodiment, an intersection may be mathematically computed using mathematical representations of the first and second borders. One of skill in the art will appreciate that any method of determining the intersection boundaries may be employed with the disclosed methods and systems. In embodiments, steps 402, 404, and 406 are repeated until the boundary for every area of interest identified by the first image recognition process is tested to see if it intersects with at least one of the boundaries of every area of interest identified by the second image recognition process. While the present embodiments have been described with respect to two image recognition processes, one skilled in the art will appreciate that one or more image recognition processes may be employed by the disclosed embodiments. In embodiments with more than two image recognition processes, steps 402, 404, and 406 are repeated until every boundary of areas of interest identified by each image recognition process are compared to each boundary of areas of interest identified by the other image recognition processes.

In embodiments, results from the voting process of method 400 may be used in confidence operation 210 (FIG. 2). For example, if method 400 determines that the boundaries of areas of interest identified by different image recognition processes intersect, the areas of interest whose boundaries intersect are assigned a higher confidence value. In further embodiments, results from the voting process of method 400 may also be used in location operation 210 (FIG. 2). For example, indications of areas of identified interest that intersect may be displayed on the image, or operation 210 (FIG. 2) may create a new area of increased interest that corresponds to the area located between the intersections determined by method 400.

FIG. 5 is an illustration of a mammogram image 500 displaying a situation where sections of two identified areas of interest overlap. FIG. 5 displays areas of interest identified (identifications 502 and 504) by two image recognition processes. Identification 502 represents an area of interest identified by a first image recognition process, as indicated by the oval boundary. Identification 504 represents an area of interest identified by a second image recognition process, as indicated by the triangle boundary. In embodiments, identifications 502 and 504 may be identified by the same image recognition process. Identifications 502 and 504 overlap, as indicated by overlapping portion 506. Because the indications overlap, there is a higher likelihood that an actual area of interest exists within the vicinity of identifications 502 and 504. For example, because overlapping portion 506 was identified as an area of interest by both the first and second image recognition processes, there is a higher probability that an actual area of interest exists at overlapping portion 506. In this embodiment, a higher confidence value should be assigned to identifications 502 and 504. In another embodiment, a higher confidence value is assigned to overlapping portion 506. In yet another embodiment, higher confidence values are assigned both to identifications 502 and 504 and overlapping portion 506. In this embodiment, the confidence assigned to each of the areas may or may not be the same (e.g., the new area of increased interest at the overlapping portion 506 may have a higher confidence than identifications 502 and 504). While embodiments of the present figure have been described using specific markings (e.g., oval and triangle boundaries), one of skill in the art will appreciate that any form of image labeling known to the art may be practiced with embodiments of the present disclosure. One of skill in the art will also appreciate that while embodiments of the present disclosure have been explained in regards to analyzing mammogram images, any type of image may be analyzed using embodiments of the present disclosure.

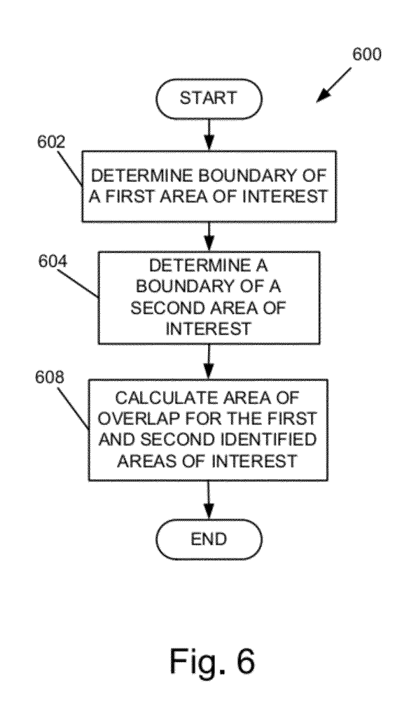

FIG. 6 is a flow chart representing an embodiment of a method 600 for applying a voting process based upon an overlap of different identified areas of interest. Flow begins at operation 602, where the method determines a boundary for a first area of interest identified by a first image recognition process. In an embodiment, the method may also determine the group of pixels representing the interior section of the area of interest as well. In one embodiment, the boundary may be determined by maintaining a list of pixels on the image corresponding to the boundary of the first identified area of interest. In embodiments, the image may be divided into nodes rather than pixels. In this embodiment, the nodes representing the identified area of interest are determined. In another embodiment, the boundary may be determined by defining a mathematical formula representing the boundary of the first identified area of interest. One skilled in the art will appreciate that any method of determining a boundary for an area of an image may be employed at operation 602.

Flow then proceeds to operation 604, where the method determines a boundary for a second identified area of interest identified by a second image recognition process. In embodiments, the second identified area of interest is identified by the second image recognition process. In another embodiment, the second identified area of interest may be identified by the image recognition process that identified the first image recognition process. Again, in one embodiment, the boundary may be determined by maintaining a list of pixels on the image corresponding to the boundary of the second identified area of interest. In an embodiment, the method may also determine the group of pixels representing the interior section of the identified area of interest as well. In embodiments, the image may be divided into nodes rather than pixels. In this embodiment, the nodes representing the area of interest may be predetermined by the initial division of the image. In another embodiment, the boundary may be determined by defining a mathematical formula representing the boundary of the second identified area of interest. One skilled in the art will appreciate that any method of determining a boundary for an area of an image may be employed at operation 604.

Once the boundaries for both areas of interest have been determined, flow proceeds to operation 606, where the method computes the intersection of the first and second identified areas of interest. In one embodiment, the determination may be made by comparing the pixels representing the first identified area of interest to the pixels representing the second identified area of interest. If the same pixel is present in both areas, the areas overlap. In another embodiment, an overlapping area, if present, may be mathematically computed using mathematical representations of the first and second borders. One of skill in the art will appreciate that any method of determining overlapping areas may be employed with the disclosed methods and systems. In embodiments, steps 602, 604, and 606 are repeated until every area of interest identified by the first image recognition process is tested to see if it overlaps with at least one of the boundaries of every area of interest identified by the second image recognition process. While the present embodiments have been described with respect to two image recognition processes, one skilled in the art will appreciate that one or more image recognition processes may be employed by the disclosed embodiments. In embodiments with more than two image recognition processes, steps 602, 604, and 606 are repeated until every area of interest identified by each image recognition process is compared to each area of interest identified by the other image recognition processes to test for overlap.

In embodiments, results from the voting process of method 600 may be used in confidence operation 210 (FIG. 2). For example, if method 600 determines that the areas of interest identified by different image recognition processes overlap, the areas of interest that overlap are assigned a higher confidence value. In further embodiments, results from the voting process of method 600 may also be used in location operation 210 (FIG. 2). For example, indications of areas of interest that intersect may be displayed on the image, or operation 210 (FIG. 2) may create a new area of interest that corresponds to the overlapping areas determined by method 600.

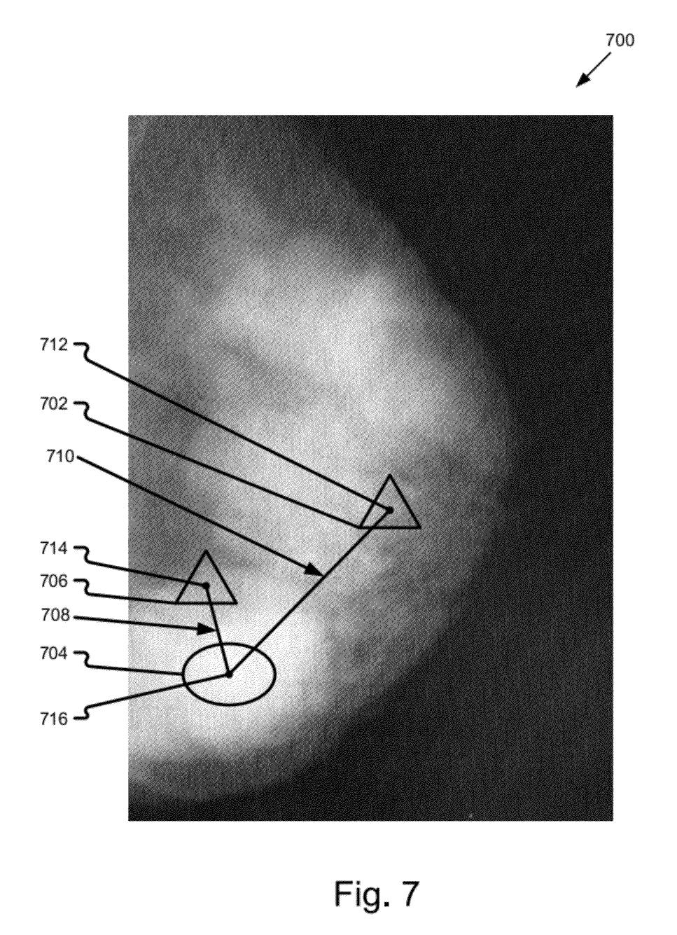

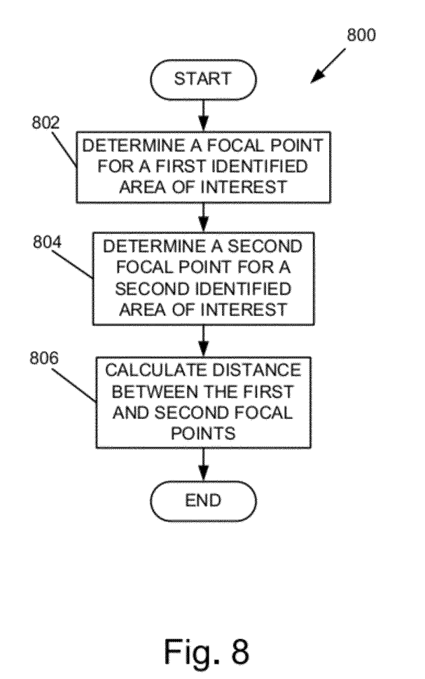

In embodiments, areas of interest may be compared by measuring differences in relative locations. In such embodiments, different measurements of relative locations may be employed (e.g., closest point on the area of interest, furthest point, a focal point as discussed further in regards to FIGS. 7 and 8, etc.)

FIG. 7 is an illustration of a mammogram image 700 displaying an embodiment where the focal points of different identified areas of interest are compared. FIG. 7 displays areas of interest identified by two image recognition processes, identifications 702, 704, and 706. Identifications 702 and 706 represent areas of interest identified by a first image recognition process, as indicated by the triangle boundary. Identification 704 represents an area of interest identified by a second image recognition process, as indicated by the oval boundary. In another embodiment, identification 704 may also be identified by the first image recognition process. In embodiments, each area of interest acknowledged by identifications 702, 704, and 706 have a focal point, e.g., focal points 712, 714, and 716. In embodiments, focal points may be the center of an area of interest. In other embodiments, focal points may be the point within an area of interest demonstrating the most interesting features or characteristics, or any other type of focal point known to the art. In embodiments, the distance between focal points may be used in determining a confidence value to assign to an area of interest. For example, a smaller the distance between focal points of two identified areas of interest may correlate to a higher confidence that the identified areas of interest correspond to actual areas of interest. This correlation is based upon the fact that two areas of interest are within a small locality. For example, the distance between the focal point 716 of identification 704 and the focal point 714 of identification 706, represented by connection 708, is relatively small. In one embodiment, identifications 704 and 706 are assigned higher confidence values because of the small distance between their respective focal points. Conversely the distance between the focal point 712 of identification 702 and the focal point 716 of identification 704, represented by connection 710, is relatively large. In embodiments, identifications 702 and 704 are assigned lower confidence values because of the large distance between their respective focal points. While embodiments of the present figure have been described using specific markings (e.g., oval and triangle boundaries), one of skill in the art will appreciate that any form of image labeling known to the art may be practiced with embodiments of the present disclosure. One of skill in the art will also appreciate that while embodiments of the present disclosure have been explained in regards to analyzing mammogram image, any type of image may be analyzed using embodiments of the present disclosure.

FIG. 8 is a flow chart representing an embodiment of a method 800 for applying a voting process based upon the comparison of focal points of different identified areas of interest. Flow begins at operation 802 where a focal point is determined for a first area of interest identified by a first image recognition process. In one embodiment, the focal point is determined using mathematical formulas for calculating the center point of an area. In another embodiment, the focal point is previously determined. In such embodiments, operation 802 gathers information related to the previously determined focal point. In yet another embodiment, the focal point may be determined by the first image recognition process, e.g., by identifying a higher concentration of interest within the area, by placing markers within the area of interest, or by any other means of identifying a focal point known in the art. In these embodiments, operation 802 again performs the task of gathering information related to the identified focal point.

Flow proceeds to operation 804 where a focal point is determined for a second area of interest identified by a second image recognition process. The second area of interest, in embodiments, is identified by the second image recognition process. In another embodiment, the second area of interest may be identified by the first image recognition process. In one embodiment, the focal point is determined using mathematical formulas for calculating the center point of an area. In another embodiment, the focal point is previously determined. In such embodiments, operation 804 gathers information related to the previously determined focal point. In yet another embodiment, the focal point may be determined by the second image recognition process or by another process, e.g., by identifying a higher concentration of interest within the area, by placing markers within the area of interest, or by any other means of identifying a focal point known in the art. In these embodiments, operation 804 again performs the task of gathering information related to the identified focal point.

After determining the focal points for the areas of interest, flow proceeds to operation 806, where the method 800 calculates the distance between the focal points. In one embodiment, the calculation may comprise counting the number of pixels or nodes along a straight line (e.g., connections 708 and 710) separating the focal points. In another embodiment the distance between the two focal points may be mathematically computed using known mathematical algorithms. One of skill in the art will appreciate that any method of calculating the distance between two points on a plane may be employed with the methods and systems disclosed herein. In embodiments, steps 802, 804, and 806 are repeated until the distances between the focal point(s) of every area of interest identified by the image recognition process and the focal point(s) of every area of interest identified by the second image recognition process have been calculated. While the present embodiments have been described with respect to two image recognition processes, one skilled in the art will appreciate that one or more image recognition processes may be employed by the disclosed embodiments. In embodiments with more than two image recognition processes, steps 802, 804, and 806 are repeated until the distance between the focal point(s) of every area of interest or identified object on the image identified by each image recognition process and the focal point(s) of each areas of interest or identified object identified by the other image recognition processes have been calculated.

While embodiments of the present disclosure have been described with reference to specific voting processes described with reference to FIGS. 3-8, one skilled in the art will appreciate that any voting process that compares the areas of interest identified by image recognition processes may be employed with embodiments of the systems and methods disclosed herein.

With reference to FIG. 9, an embodiment of a computing environment for implementing the various embodiments described herein includes a computer system, such as computer system 900. Any and all components of the described embodiments may execute on a client computer system, a server computer system, a combination of client and server computer systems, a handheld device, and other possible computing environments or systems described herein. As such, a basic computer system applicable to all these environments is described hereinafter.

In its most basic configuration, computer system 900 comprises at least one processing unit or processor 904 and system memory 906. The most basic configuration of the computer system 900 is illustrated in FIG. 9 by dashed line 902. In some embodiments, one or more components of the described system are loaded into system memory 906 and executed by the processing unit 904 from system memory 906. Depending on the exact configuration and type of computer system 900, system memory 906 may be volatile (such as RAM), non-volatile (such as ROM, flash memory, etc.), or some combination of the two.

Additionally, computer system 900 may also have additional features/functionality. For example, computer system 900 includes additional storage media 908, such as removable and/or non-removable storage, including, but not limited to, magnetic or optical disks or tape. In some embodiments, software or executable code and any data used for the described system is permanently stored in storage media 908. Storage media 908 includes volatile and non-volatile, removable and non-removable media implemented in any method or technology for storage of information such as computer readable instructions, data structures, program modules, or other data. In embodiments, images, such as mammogram images, and/or the various image recognition processes and voting processes are stored in storage media 908.

System memory 906 and storage media 908 are examples of computer storage media. Computer storage media includes, but is not limited to, non-transitory storage media, such as RAM, ROM, EEPROM, flash memory or other memory technology, CD-ROM, digital versatile disks (DVD) or other optical storage, magnetic cassettes, magnetic tape, magnetic disk storage, other magnetic storage devices, or any other medium which is used to store the desired information and which is accessed by computer system 900 and processor group 904. Any such computer storage media may be part of computer system 900. In some embodiments, images, such as mammogram images, the various image recognition processes and voting processes, and/or the results generated by the various processes, systems, and methods are stored in system memory 906. In embodiments, system memory 906 and/or storage media 908 stores data used to perform the methods or form the system(s) disclosed herein, such as image data, mathematical formulas, image recognition processes, voting processes, etc. In embodiments, system memory 906 would store information such as image data 920 and process data 922. In embodiments, image data 920 may contain actual representations of an image, such as a mammogram image 100 (FIG. 1). Application data 916, in embodiments, stores the procedures necessary to perform the disclosed methods and systems. For example, application data 922 may include functions or processes for image recognition or voting, functions or processes for displaying the identified areas of interest, etc.

Computer system 900 may also contain a processor, such as processor P1 914. Processor group 904 is operable to perform the operations necessary to perform the methods disclosed herein. For example, processor group 904 may perform the operations of the various image recognition processes and voting processes. In one embodiment, processor group 904 may comprise a single processor, such as processor P1 914. In other embodiments, processor group 904 may comprise multiple processors, such as processors P1 914, P2 916, and Pn 918, such as in a multiprocessor system. One of skill in the art will recognize that any number of processor may comprise processor group 904. In embodiments utilizing a multiprocessor environment, each processor of the multiprocessor environment may be dedicated to process the computations of a specific image recognition process. In such an embodiment, image recognition processes may be performed in parallel, leading to an efficient distribution of processing power as well as an increase in processing time for the various systems and methods disclosed herein. In further multiprocessor embodiments, specific processors may be dedicated to process the computations involved in the various comparisons and voting processes. In yet another embodiment, similar tasks performed by different image recognition processes can be grouped together and processed by a processor dedicated to processing such a task. One skilled in the art will appreciate that any method, process, operation, or procedure disclosed herein may be individually processed by a dedicated processor.

Computer system 900 may also contain communications connection(s) 910 that allow the device to communicate with other devices. Communication connection(s) 910 is an example of communication media. Communication media may embody a modulated data signal, such as a carrier wave or other transport mechanism and includes any information delivery media, which may embody computer readable instructions, data structures, program modules, or other data in a modulated data signal. The term “modulated data signal” means a signal that has one or more of its characteristics set or changed in such a manner as to encode information or a message in the data signal. By way of example, and not limitation, communication media includes wired media such as a wired network or direct-wired connection, and wireless media such as an acoustic, RF, infrared, and other wireless media. In an embodiment, mammogram images and or determinations of probability results may be transmitted over communications connection(s) 910.

In embodiments, communications connection(s) 910 may allow communication with other systems containing processors. In such an embodiment, a distributed network may be created upon which the disclosed methods and processes may be employed. For example, image recognition processes may be divided along the distributed network such that each node, computer, or processor located on the network may be dedicated to process the calculations for a single image recognition process. In such an embodiment, image recognition processes may be performed in parallel, leading to an efficient distribution of processing power as well as an increase in processing time for the various systems and methods disclosed herein. In further distributed network embodiments, specific computers, nodes, or processors located on the network may be dedicated to process the computations involved in the various comparisons and voting processes disclosed herein. One skilled in the art will appreciate that any method, process, operation, or procedure disclosed herein may be individually processed by a dedicated computer, node, or processor in a distributed network.

In some embodiments, computer system 900 also includes input and output connections 912, and interfaces and peripheral devices, such as a graphical user interface. Input device(s) are also referred to as user interface selection devices and include, but are not limited to, a keyboard, a mouse, a pen, a voice input device, a touch input device, etc. Output device(s) are also referred to as displays and include, but are not limited to, cathode ray tube displays, plasma screen displays, liquid crystal screen displays, speakers, printers, etc. These devices, either individually or in combination, connected to input and output connections 912 are used to display the information as described herein. All these devices are well known in the art and need not be discussed at length here.

In some embodiments, the component described herein comprise such modules or instructions executable by computer system 900 that may be stored on computer storage medium and other tangible mediums and transmitted in communication media. Computer storage media includes volatile and non-volatile, removable and non-removable media implemented in any method or technology for storage of information such as computer readable instructions, data structures, program modules, or other data. Combinations of any of the above should also be included within the scope of readable media. In some embodiments, computer system 900 is part of a network that stores data in remote storage media for use by the computer system 900.

In other embodiments, one or more voting functions are applied to result sets derived from one or more image recognition processes in order to more accurately identify areas of interest. In such embodiments, an area of interest comprises a hypothesis about the significance of a particular portion of image data. The image recognition processes may be designed to identify such areas of interest based on a variety of criteria. Thus, different image recognition processes may produce different result sets identifying different areas of interest. Furthermore, each recognition process may have a different level of confidence attached to their respective results. Applying one or more voting functions effectively combines the different result sets resulting in a final result set that more accurately identifies areas of interest than individual recognition processes on their own.

A voting function is a process or function that may be applied to a data from one or more image recognition processes. The voting function improves the accuracy of the data by allowing each hypothesis that comprises an identified area of interest to be affected by the presence or absence of other areas of interest in an image. A voting function may be applied to a set of data points identified as areas of interest on an image by a single image recognition process or by multiple image recognition processes. The voting function may be used to confirm actual areas of interest or filter out false positive identifications from the set(s) of data. For example, applying a voting function may increase or decrease the amplitude of an identified area of interest based on the proximity of that area of interest to other identified areas of interest. Furthermore, a voting function may create areas of interest not present in the set of the areas of interest identified by the image recognition processes. A voting function may take a variety of forms. In embodiments, representations of areas of interest are created by transforming a set of data points into continuous fuzzy set membership functions. The voting function may then comprise, for example, calculating a superposition of those continuous functions using fuzzy logic operations.

Representations of areas of interest may depend on the information provided by an image recognition process. For example, an image recognition process provides a confidence value for an identified area of interest. The representations of the areas of interest may be created by calculating pyramid-like or Gaussian function of image coordinates centered at a focal point of the area of interest and having an amplitude calculated as a monotonically increasing function of the area's confidence. Although specific examples of functions are discussed, one of skill in the art will appreciate that additional functions may be practiced with the systems and methods disclosed herein.

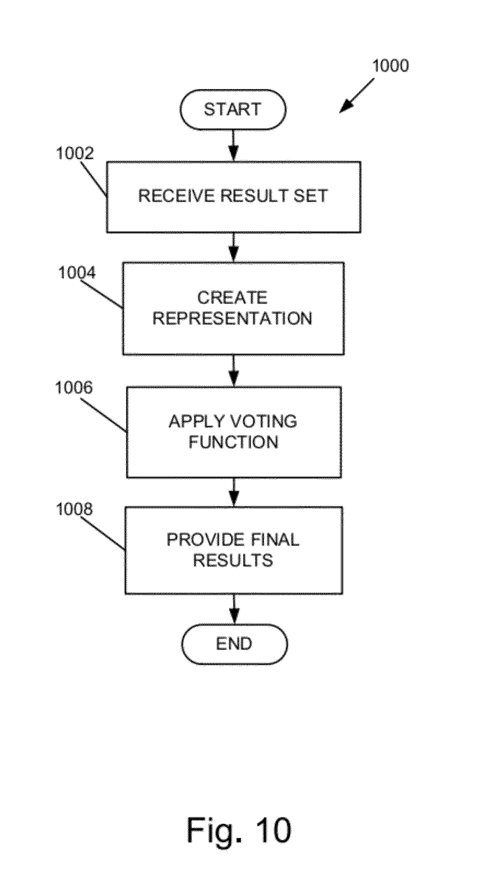

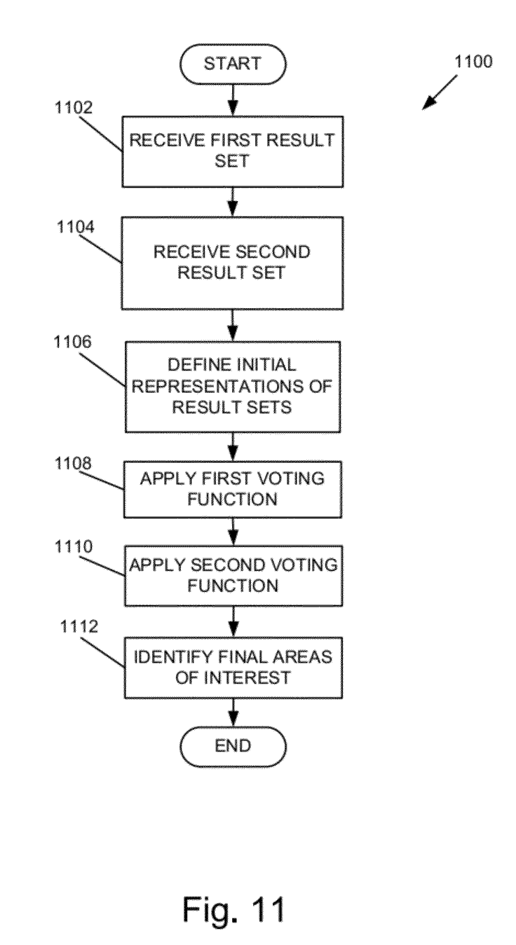

Embodiments of applying a voting function to one or more result sets derived from one or more image recognition processes are now described in more detail with respect to FIGS. 10 and 11. FIG. 10 is a flow chart representing an embodiment of a method 1000 for applying a voting function to a result set from an image recognition process. Flow begins at operation 1002, where a result set is received from an image recognition process. In one embodiment, the result set may be received by another process or application separate from the image recognition process. In an alternate embodiment, the result set may be received by another function or process residing within the same application performing the image recognition process. The result set may include a set of one or more areas of interest initially identified by the image recognition process. Furthermore, the result set may be an empty set indicating that the image recognition process did not identify any initial areas of interest. The areas of interest may be identified as specific coordinates on the image, as a region on the image, as a function of image coordinates which reflects the probability of a lesion at a given point, or any other type of identification employed by the image recognition process to identify areas of interest. One of skill in the art will recognize that the initial results in the result set may take the form of any type of indication, identification, etc. used in the art.

In further embodiments, other information, such as the confidence values associated with the areas of interest, the type of area of interest (mass, microcalcification, architectural distortion, etc.), breast density, breast contour, location of muscle, location of the nipple, location of vessels, degree of calcification in vessels, etc. may be received from the image recognition process at operation 1002. For example, each individual result in the result set may have a separate confidence value associated with it based on its individual area of interest. In other embodiments, a single confidence value may be applied to each result in the result set, for example, if the image recognition process has a certain confidence associated with its performance. The confidence values may be generated and sent by the image recognition process. In other embodiments, the image recognition process may not send information related to confidence. In such embodiments, a separate application or process, such as the application or process performing the method 1000, may assign the confidence value to the individual results or the result set. In still other embodiments, input confidence values may be unnecessary.

Flow proceeds to operation 1004, where the method 1000 creates a representation of the result set. The representation may be a continuous function based upon the image coordinates. For example, the representation created at operation 1004 may be a continuous function ƒ of the image coordinates x and y (where x and y represent coordinates along the x- and y-axis respectively) such that the representation of the result set is f(x, y). One of skill in the art will recognize that the representation may be built by using a different type of function ƒ which may operate on input other than the x- and y-axis coordinates. For example, any of the output derived by the image recognition process may be operated upon by the function ƒ. The function ƒ(x, y) is created such that the region of the function's maximum approximately corresponds to the location of an initial area of interest identified in the received result set. If the original result set has more than one initial area of interest, the function ƒ(x, y) may have a similar number of local maxima such that each local maximum corresponds to an initial area of interest identified in the result set or, in other embodiments, separate functions may be created to represent each initial area of interest. For example, an area of interest may be represented by f(x, y) being a triangular function, a piecewise polynomial function centered at a focal point for an initial area of interest, or any type of function capable of producing a reliable representation of the identified areas of interest. Furthermore, the amplitude of the one or more local maxima of the function ƒ(x, y) may be a monotonic increasing function g(c) based on one or more confidence values c associated with image recognition process or with the initial areas of interest identified in the result set. In one embodiment, the function g(c) may be specific to the image recognition process. In other embodiments, the function g(c) may be specific to the result set or the individual results (e.g., the initial areas of interest identified in the result set). For example, g(c) may be a linear function, a sigmoid function, an exponentiation function, or any other monotonic function of the confidence value c associated with the one or more initial areas of interest. While the disclosure details specific representations that may be created at operation 1004, one of skill in the art will recognize that the systems and methods disclosed herein are not limited to such specific representations. Indeed, the embodiments disclosed herein may be practiced with any type of representation known to the art, or even no representation at all.

Flow proceeds to operation 1006 where a voting function is applied to the result set. In one embodiment, the voting function is applied to the one or more representations created at operation 1004. In another embodiment, the voting function is applied to the result set itself or the one or more initial areas of interest identified in the result set. As previously described, a voting function is a process or function that may be applied to data from an image recognition process or any other type of identification process. The voting function improves the accuracy of the data by allowing each hypothesis that comprises an identified area of interest to be affected by the presence or absence of other areas of interest in an image.

After applying the voting function at operation 1006, flow proceeds to operation 1008 where the method 1000 produces the final results of the voting process. The final results may be a model representing final areas of interest produced by applying the voting function to the initial result set received at operation 1002 or the representation created at operation 1004. The final model may be a function based on image coordinates W(x, y) such that the local maxima of the function W(x, y) correspond to final areas of interest on the image. In such embodiments, the is final areas of interest are identified by calculating the local maxima of the function W(x, y). In another embodiment, a final result set may be provided instead of a function W(x, y) at operation 1008. In still other embodiments, the identification of final results in operation 1008 may be considered as a specific example of determining a “confidence value” for the final areas of interest. Because of the voting function, the final areas of interest provided at operation 1008 are more likely to identify actual areas of interest, that is, the final areas of interest have a higher likelihood of correspond to something of actual interest such as, for example, a tumor or lesion in a mammography image. The final results, whether in the form of a model function W(x, y), a result set, or otherwise can then be used by humans (e.g., doctors, technicians, etc.) or other applications to aid in the identification of cancer, lesions, calcifications, tumors, cysts, or other ailments found in medical images.

FIG. 11 is a flow chart representing an embodiment of a method 1100 for applying voting functions to multiple result sets from multiple image recognition processes. While the specific embodiment illustrated in the method 1100 relates to two result sets from two image recognition processes, one of skill in the art will recognize that any number of result sets and image recognition processes may be employed with the method 1100. Similarly, the method 1100 may be practiced using a single image recognition process, for example, by deriving separate result sets from a single image recognition process by running the image recognition process twice with different settings, inputs, etc.

Flow begins at operation 1102 where the method 1100 receives a first result set from a first image recognition process. The first result set may be received by a process, application, and/or machine performing the method 1100 that is separate from the image recognition process. In an alternate embodiment, the first result set may be received by another function or process residing within the same application, process, and/or machine performing the image recognition process. The first result set includes a set of one or more areas of interest initially identified by the image recognition process. Additionally, the result set may be an empty set indicating that the image recognition process did not identify any initial areas of interest. The areas of interest may be identified as specific coordinates on the image, as a region on the image, as a function of image coordinates which reflects the probability of a lesion at a given point, or any other type of identification employed by the image recognition process to identify areas of interest. One of skill in the art will recognize that the initial results in the result set may include any type of other data used in the art such as, for example, confidence values.

Flow proceeds to operation 1104 where the method receives a second result set identifying second initial areas of interest from a second image recognition process. The second result set may be an empty set or a set of one or more initial areas of interest identified by the second image recognition process. In another embodiment, the second result set may be derived by the first image recognition process run in a different operating condition (e.g., adjusted settings, different input, etc). As with the first result set, one of skill in the art will appreciate that any type of data represented in any form may be received by the method 1100 at operation 1104.

Upon receiving the first and second result sets, flow proceeds to operation 1106 where initial representations are defined for the initial areas of interest identified in the first and second result set. In other embodiments, operation 1106 may be skipped and flow may proceed to operation 1108 where the first voting function is applied directly to the first and second result sets. In one embodiment, a continuous function ƒ is defined for each initial area of interest in the first and second result sets based on image coordinates x and y (where x and y represent coordinates along the x- and y-axis respectively). One of skill in the art will recognize that the function ƒ may operate on input other than the x- and y-axis, for example, any of the output derived by the image recognition process may be operated upon by the function ƒ or any other functions disclosed herein. The continuous function ƒ(x, y) is defined such that its one or more maxima approximately correspond to the one or more locations of the initial areas of interest defined in the first and second result sets. For example, for an initial area of interest, f(x, y) may be, for example, a pyramid-like

$$f ( x , y ) = max ( 0 , 1 - \frac{1}{r} max ( \vert x - {x}_{c} \vert , \vert y - {y}_{c} \vert ) )$$

centered at a focal point of this area of interest. One of skill in the art will appreciate that the method is not limited to pyramid-like function representations. Rather, any type of functional representation may be practiced with the present disclosure.

Continuing with the previous example, the amplitude of the one or more local maxima f(x, y) may be a monotonic increasing function g(c), where c is an input confidence value. The input confidence values may be received along with the result sets from one or more image recognition processes in operations 1102 and 1104, or may be separately determined by the method 1100. For example, the method 1100 may assign a confidence level to a result set based upon the level of trust that the method ascribes to the particular image recognition process that produced the result set, thus making the confidence values specific to a particular image recognition process. One way of establishing confidence values for each image recognition process may be by assigning confidence value as monotonic function of sensitivity level of the recognition process in relation to a certain false positive level of the recognition process. Another way of establishing confidence values for each image recognition process may be by introducing parameters for each recognition process representing the confidence values of recognition processes and then optimizing these parameters on an image set by selecting parameters which maximize the final results of the voting function. The optimization can be done by any well-known optimization methods such as, but not limited to, a Monte-Carlo method. Additionally, confidence values for each image recognition process can depend on the characteristics of the examined body part (e.g., breast) or area of interest. For example, the level of trust ascribed by the method to the image process can depend on breast density or the size of area of interest. As an example, the function g(c) may be a linear function, a sigmoid function, an exponentiation function, or any other monotonic function of the confidence c of the initial areas of interest. One of skill in the art will appreciate that although the disclosure recites specific types of functions as representations of the initial areas of interest in the first and second result set, the representations may be defined by other functions or by any other means that can be employed to represent the initial areas of interest. Furthermore, while operation 1102 is described as creating a representation for each initial area of interest in the first and second result sets, in other embodiments a single representation may be defined for all the areas of interest within a result set.

After defining the initial representations at operation 1106, flow proceeds to operation 1108 where a first voting function is applied to the initial representations. As previously described, a voting function is a process or function that may be applied to a data from one or more image recognition processes. The voting function improves the accuracy of the data by allowing each hypothesis that comprises an identified area of interest to be affected by the presence or absence of other areas of interest in an image. A voting function may be applied to a set of data points identified as areas of interest on an image by a single image recognition process or by multiple image recognition processes. For example, the first voting function applied to the initial representations at operation 1108 may calculate a function F of image coordinates x and y such that F(x, y) is a superposition of the functions ƒ(x, y) defined at operation 1106. For example, F(x, y) may be calculated by combining the functions ƒ(x, y) using some fuzzy logic operation. In one embodiment the bounded sum t(p, q)=min(1, p+q), where p and q are values of the functions ƒ(x, y) corresponding to the first and second initial representations defined at operation 1006, may be used to calculate F(x, y). In other embodiments, a separate function F(x, y) will be calculated for each of the first and second result set such that operation 1108 will result in two functions F(x, y) and F′(x, y) that represent all of the initial areas of interest identified in the first and second result sets, respectively. In embodiments, calculating the function F(x, y) results in a composite representation of all the initial areas of interest in a result set. The composite representation further provides the benefit of allowing each initial area of interest to affect each of the other representations in a way that increases the accuracy of the identified areas of interest. As an example, if there are two initial areas of interest located near each other on the image, application of the first voting function may result in a composite representation in which the two areas of interest are more prominently displayed due to the fact that the proximity of the individual areas of interest increases the likelihood that an actual area of interest is present in their region. For example, the resulting composite representation F(x, y) may result in higher amplitudes at the local maxima representing the two initial areas of interest than the original representations f(x, y) defined at operation 1106. Conversely, two or more initial area of interest may have a negative effect on each other and therefore lower the amplitudes of the local maxima in F(x, y). In alternate embodiments, if a single function was defined to represent an entire result set at operation 1106, the first voting process applied to the initial representations at 1108 may perform an operation other than calculating a superposition or may be skipped entirely.

Upon calculating the composite representations at operation 1108, flow proceeds to operation 1110 where a second voting function is applied to the composite representations. Continuing the previous example, the second voting function may calculate a function W(x, y) that is a superposition of the functions F(x, y). In an embodiment, W(x, y) is created by combining the functions F(x, y) using a fuzzy logic operation. For example, the minimum function t(p, q)=min(p, q), a nilpotent maximum or other t-norm function, an Einstein sum, etc. may be used. Here, p and q correspond to the values of F(x, y) and F′(x, y) corresponding to the first and second initial representations at a given point on the image. The composite function W(x, y) represents a unified composite model of the initial areas of interest in the first and second result sets identified by the first and second image recognition processes, respectively. Similar to the process described in operation 1108, the areas of interest represented by local maxima for the initial areas of interest in the first and second result sets defined by the composite representations F(x, y) have an effect on each other when combined using fuzzy logic operations. Local maxima in the same region of the functions F(x, y) have a positive effect on each other which is represented, for example, by an increase in the amplitude of local maxima in the corresponding regions of the unified composite model W(x, y). Conversely, the lack of proximity of local maxima in the functions F(x, y) has a negative effect on the amplitude of the local maxima in the resulting composite model. Thus, applying the second voting function at operation 1110 combines the results from multiple image recognition processes thereby increasing the accuracy of correctly identifying actual areas of interest.

Upon applying the second voting function and creating the unified composite model at step 1110, flow proceeds to operation 1112 where final areas of interest are identified using the unified composite model (e.g., W(x, y)). The final areas of interest are identified by finding all of the local maxima of the function W(x, y). Furthermore, regions of interest corresponding to the final areas of interest can be identified from the composite model by analyzing the behavior of W(x, y) in the vicinity of the local maxima. For example, a region may be identified as a set of x and y coordinates such that W(x, y)>=k, where k is a constant that may or may not depend on a particular maximum. For example, k can be a predetermined value or percentage of the amplitude of the local maximum. This value may also depend on the hypothesis type (e.g., mass, microcalcification, architectural distortion, etc.) and/or on image type (e.g., breast density, the degree of vessel calcification, etc.) Furthermore, confidence value of the final area of interest can be calculated using the amplitude of the corresponding maximum of W(x, y) and other available data. In other embodiments, the calculation of confidence values of the final areas of interest might be unnecessary.

As previously described, the final areas of interest have a higher likelihood of identifying actual areas of interest by virtue of the application of the various voting functions. For example, the voting functions described herein first combine the results in each individual result set in a manner that the initial areas of interest had an effect on each other. Furthermore, composite models created during the first voting process are then combined with further effect upon the identified areas, resulting in a comparison between the results from multiple image recognition processes, thereby increasing the accuracy of the final areas of interest derived from the unified composite model. Upon identifying the final areas of interest at operation 1112, the final areas of interest may be provided to another application, program or function, or be displayed to a user.

An illustration of an embodiment of the method and system at work will aid in more fully understanding an embodiment of the present disclosure. The following description is intended to provide an example of an embodiment of the disclosure and not to limit the disclosure in any way. An application residing on a computer system, such as computer system 900 is used to analyze mammogram images to identify areas of interest on the image. In embodiments, areas of interest may be portions of the image displaying instances of cancer, lesions, calcifications, tumors, cysts, or other ailments. An image, such as a mammogram image 100 is inputted into the application. In embodiments, the application then applies a plurality of image recognition processes to analyze the image. One of skill in the art will appreciate that the number of image recognition processes applied to the image is irrelevant so long as at least one unique image recognition process is applied. Each image recognition process applied may identify areas of interest on the mammogram image independently, e.g., without sharing information with other image recognition processes or based solely upon the determinations of an individual image recognition process. In other embodiments, the image recognition processes may work together to identify different areas of interest. In embodiments, each image recognition process is processed by a dedicated processor in a multiprocessor system or over a distributed network, thereby allowing the image recognition processes to be processed in parallel, thus increasing computational efficiency and spreading the workload across multiple processors.

In embodiments, after the image recognition processes individually identify areas of interest or objects on the mammogram image, the different identified areas of interest or objects are compared to determine a confidence value related to the accuracy of the identifications. In embodiments, the comparison is done using a voting process. Comparing the results of multiple image recognition processes allows for the mitigation of the inherent faults of the image recognition process, thus leading to reduced false positive and false negative rates. Additionally, methods utilizing multiple image recognition processes, rather than a single one, amicably lend themselves to multiple processor systems or networks. On the other hand, developing a more complicated image recognition process does not necessarily ensure that the image recognition process is free from inherent faults, nor does a single, more complicated process lend itself to a multiprocessor system or network due to the difficulty in dividing a single process among several processors. Thus, embodiments of the disclosed methods and system(s) provided for increased accuracy and computation efficiency. While embodiments of the present disclosure have been explained in regards to analyzing a mammogram image, one of skill in the art will appreciate that any type of image may be analyzed using embodiments of the present disclosure.

In embodiments, the results of the comparison are used in determining confidence values for the areas of interest. In embodiments, indications of areas of increased interest with a confidence value over a certain threshold are displayed on the mammogram image. In other embodiments, the results of the comparison may also be used in calculating new areas of interest. In embodiments, the new areas of interest may be a combination of areas of interest identified by separate image recognition processes.

In embodiments, indications of areas of increased interest are displayed on the mammogram image, and the image is then displayed for human analysis. In embodiments, the mammogram image containing indications of areas of interest may be displayed on a computer monitor or printed in some form for human analysis. In such embodiments, the disclosed methods and system(s) may be used to aid physicians in detecting cancer. In other embodiments, the information related to the areas of interest is stored for operation by another application.

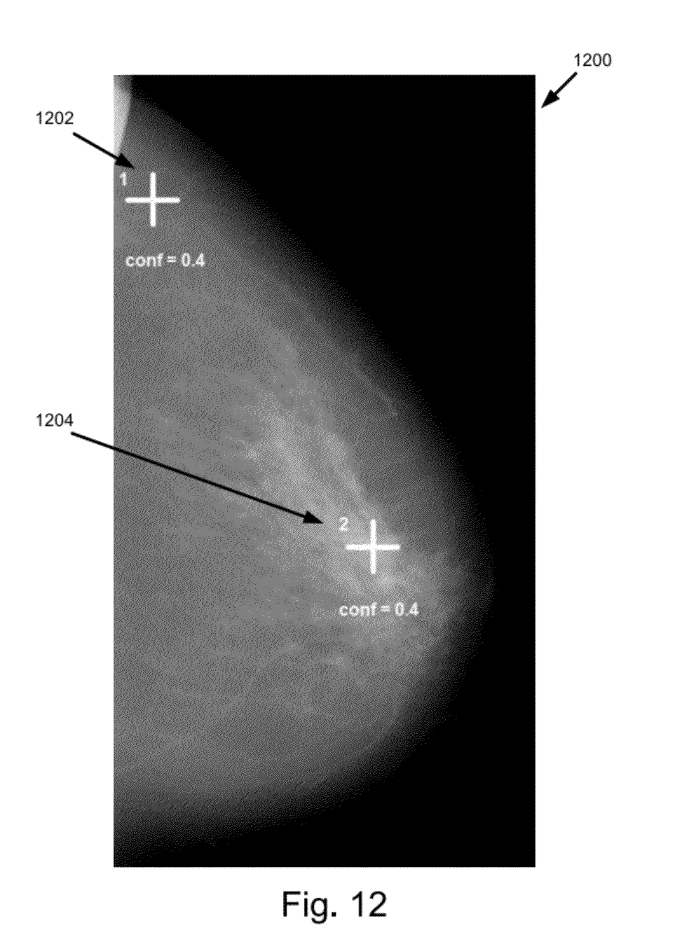

FIGS. 12-19 will now be referenced in order to provide an example of applying the previously discussed voting functions to medical images (e.g., mammogram images). While FIGS. 12-19 provide specific illustrations and examples of applying voting functions, one of skill in the art will appreciate that the figures and corresponding discussion are intended to provide additional clarification of the systems and methods disclosed herein and in no way limit the scope of the present disclosure. The principles discussed herein readily apply to uses other than those related to mammography or medical imaging in general. Rather these examples are intended to provide an example use of the systems and methods disclosed herein.

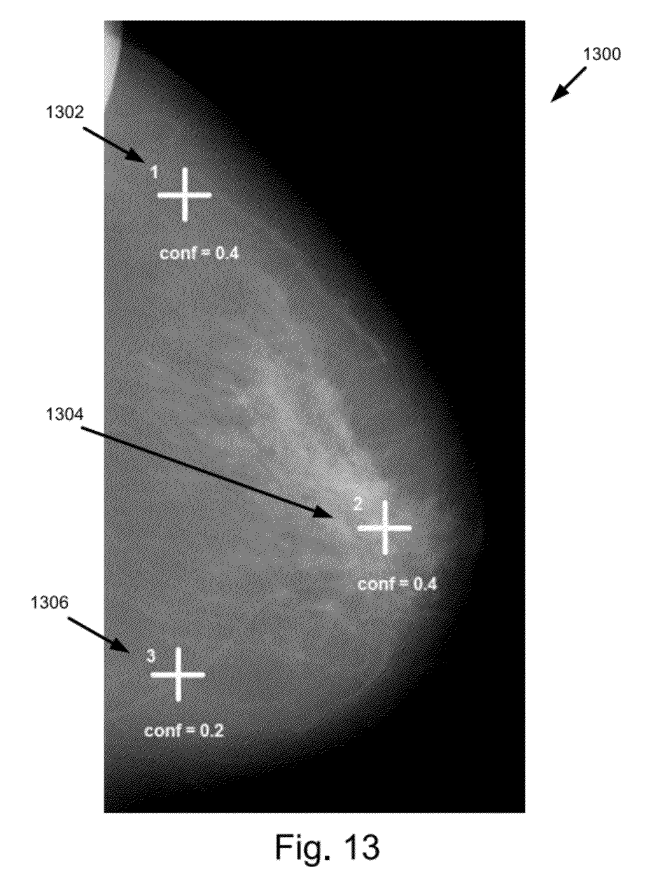

Proceeding with the example, a mammography image is analyzed by two image recognition processes. The output from each image recognition process is a set of coordinates. The coordinates specify the location of an initial area of interest identified by each recognition process. Furthermore, in this example, each image recognition process also provides a confidence value that is associated with each initial area of interest. FIG. 12 is an illustration 1200 of initial areas of interest 1202 and 1204 identified by a first image recognition process on a mammogram image 1200. FIG. 13 is an illustration 1300 of initial areas of interest 1302, 1304, and 1306 identified by a second image recognition process on the same mammogram image 1200.

In the provided examples, the initial areas of interest 1204 and 1304 identified by the first and second recognizers, respectively, are correctly identified. The initial areas of interest 1202, 1302, and 1306 are incorrectly identified by the first and second image recognition processes. However, the first image recognition process assigns the same confidence value to the incorrectly identified initial area of interest 1202 as the correctly identified area of interest 1204. Similarly, the second image recognition process assigns the same confidence value to the incorrect initial area of interest 1302 as the correctly identified area 1304. As will be demonstrated, the voting processes disclosed herein provide an easier way to correctly identify areas of interest that can also help deal with the confidence value problems shown in FIGS. 12 and 13.

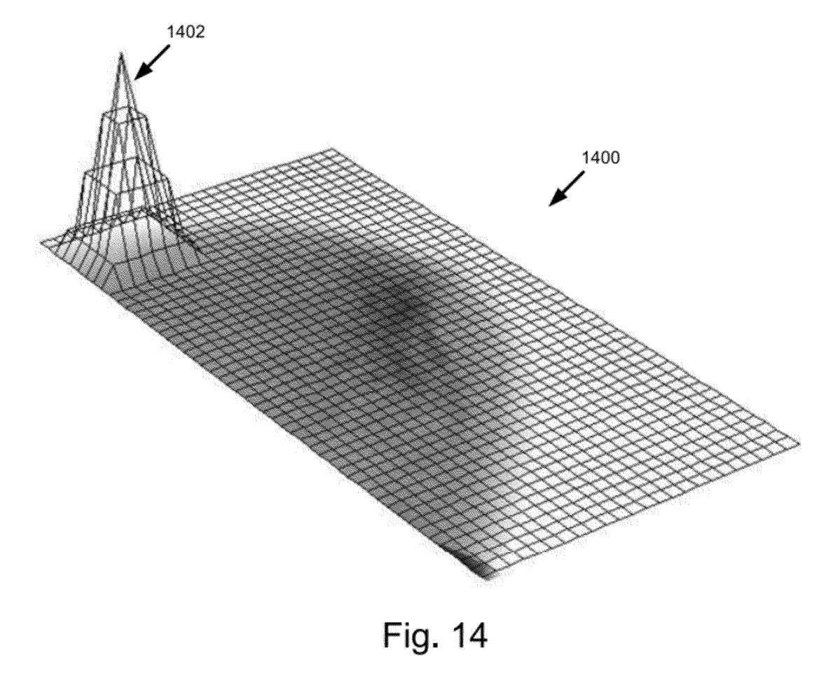

As described with respect to steps 1004 and 1106, representations of the initial areas of interest are constructed. As an example, a continuous representation is created for each initial area of interest returned by each image recognition process in the form of a functions ƒ(x, y). FIG. 14 is an example illustration 1400 of a continuous representation 1402 of the first initial area of interest 1202 (FIG. 12) identified by the first image recognition process. In the example, a pyramid-like function

$$f ( x , y ) = C max ( 0 , 1 - \frac{1}{r} max ( \vert x - {x}_{c} \vert , \vert y - {y}_{c} \vert ) )$$

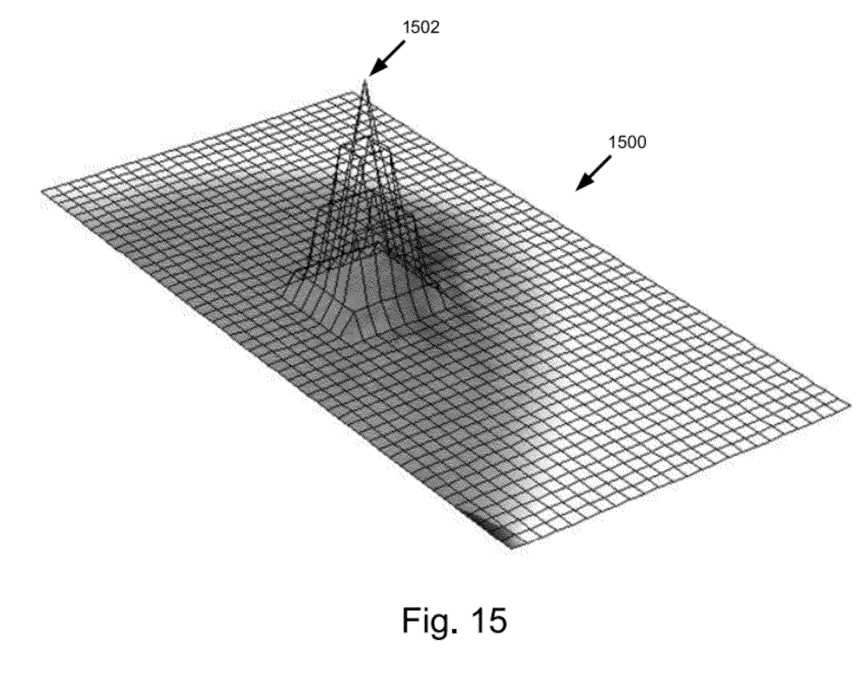

is centered at the point of interest to create the continuous representation 1402. Here, C is a confidence value associated with the initial area of interest (e.g., 1202), r is a constant which specifies how wide the ‘pyramid’ is, and x, and y, are the coordinates of the location. As is shown, the plane of the illustration 1400 corresponds to the image processed by the image recognition process. Furthermore, as shown, the plane of the illustration 1400 may be shaded to indicate which portions of the image are covered by the breast. The continuous representation 1402 is located at the same coordinates on the plane of the illustration 1400 as the coordinates of the initial area of interest 1202 identified by the first image recognition process on the image 1200. A continuous representation 1502 of the second initial area of interest 1204 is similarly constructed and shown in illustration 1500 of FIG. 15. Although not shown, continuous representations of the three initial areas of interest 1302, 1304, and 1306 identified by the second image recognition process may be similarly constructed.

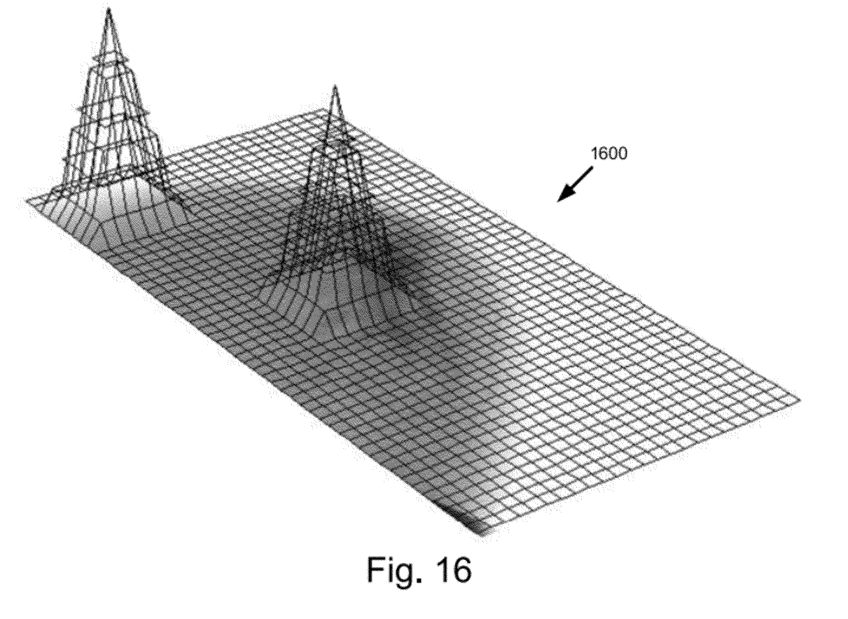

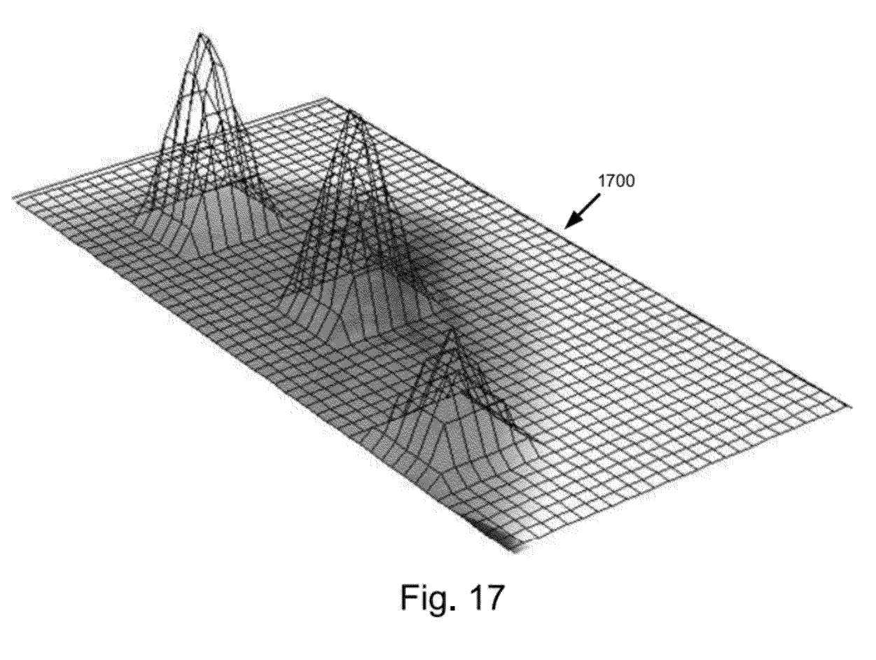

After the construction of the continuous representations (e.g., representations 1402 and 1502), a first voting function is applied to the continuous representations to create a combined representation F(x, y) for the initial areas identified by the first recognition process and the second image recognition process, respectively. The combined representation F(x, y) corresponds to the whole set of answers returned by an individual image recognition process. It is calculated by combining the previously constructed representations f(x, y). For example, a bounded sum function t(p, q)=min(1, p+q) may be used to calculate a superposition of the functions ƒ(x, y). FIG. 16 provides an example illustration 1600 of combined representation F(x, y) for the first image recognition process with both continuous representations 1402 and 1502. Note that although in this example, the example illustration 1600 appears to have simply added the continuous representation 1402 to the same plane of illustration as the continuous representation 1502, this is not necessarily the case. Depending upon the functions defined for a particular implementation, the construction of the combined representation F(x, y) (e.g., illustration 1600) may cause the previously constructed representations f(x, y) to be influenced by one another. FIG. 17 provides an example illustration 1700 of combined representation F(x, y) for the second image recognition process (combining the constructed representations f(x, y) for the areas of interest 1302, 1304, and 1306 from FIG. 13).

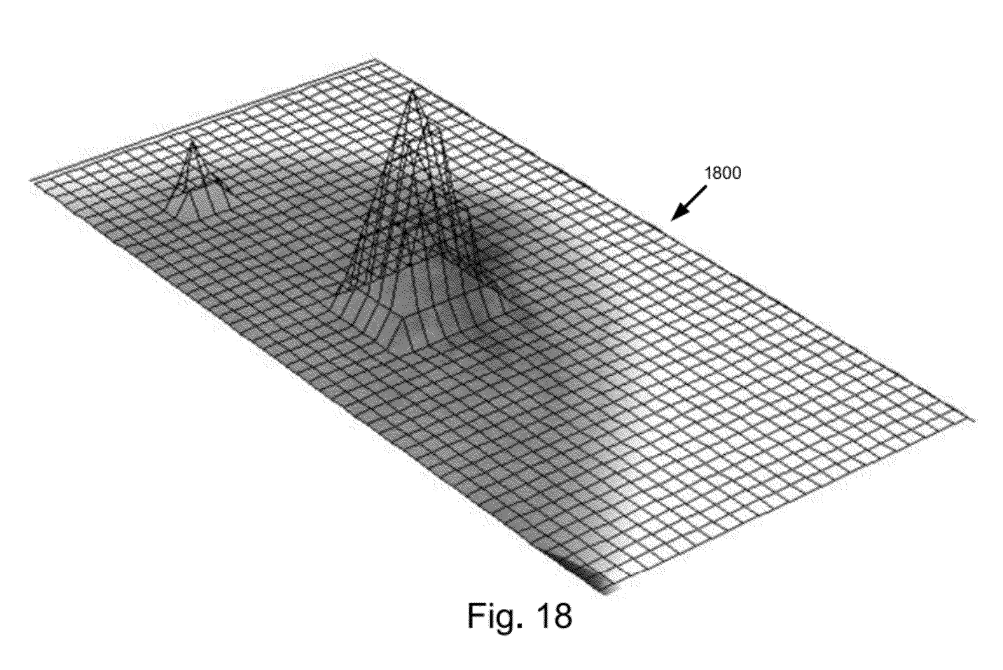

After creating the combined representations F(x, y), a second voting function is applied to the combined representations 1600 and 1700 to create a unified composite model W(x, y). For example, the unified composite model W(x, y) may be created with a minimum function t(p, q)=min(p,q) to combine the two combined representations F(x, y) into W(x, y). FIG. 18 is an embodiment of an illustration 1800 of a unified composite model W(x, y) of the results from the first and second image recognition processes used in this example. By applying the various voting functions to combine and compare the individual areas of interest, the amplitudes of the different areas of have changed in comparison to the first representations created, and in some cases representations of initial areas of interest may not even be included in the unified composite model (e.g., there is no indication of the initial area of interest 1306).

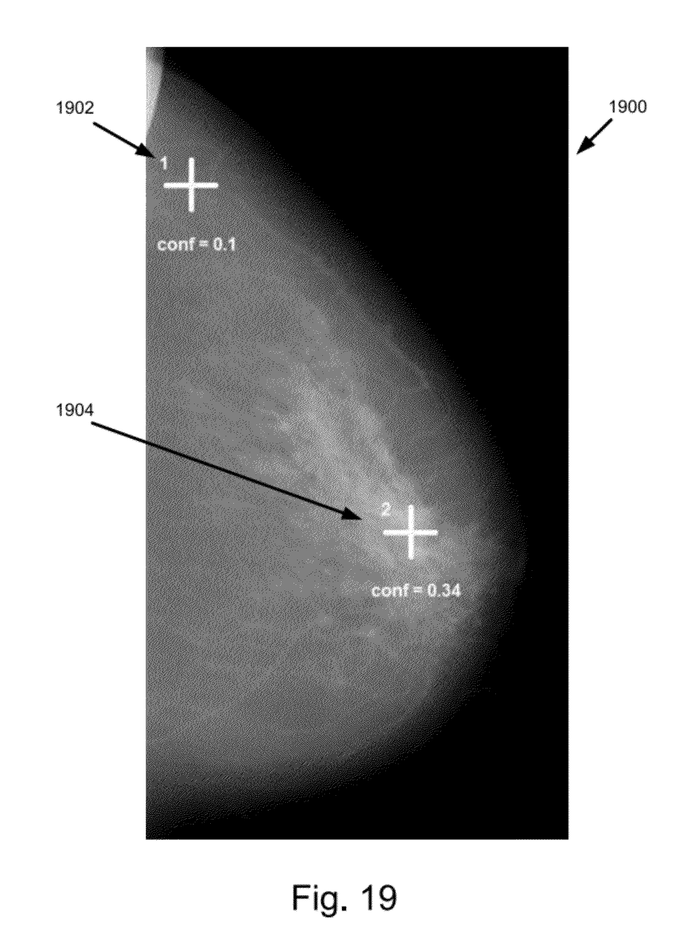

Upon creating the unified composite model, final areas of interest can be identified by finding all of the local maxima of the unified composite model W(x, y). The final confidence value for each location is calculated as the amplitude of the corresponding local maximum of W(x, y). Thus, by applying the voting functions, the final results can be outputted as provided by the example result 1900 of FIG. 19. As demonstrated in FIG. 19, the two correctly identified locations 1204 and 1304 were combined into a single representation 1904, one of the false locations 1306 was completely eliminated, and the other two false locations 1202 and 1302 were combined into a single representation 1902 with a confidence value that is significantly lower than the confidence value of the two initially identified false locations 1202 and 1302. Thus, by applying the voting functions, the results identified by image recognition processes are made more reliable and lead to more accurate identification of areas of interest in image processing. This is especially useful for medical image processing, such as the example provided illustrating the detection problem areas in mammogram images.

In further embodiments, one or more voting processes may be applied to result sets received from a user to help identify areas of interest in an image. In such embodiments, the user's identified areas of interest may be used in conjunction with one or more sets of areas of interest identified by one or more image recognition processes in order to provide more accurate results by applying a voting process to the user results set and the one or more image recognition process result sets. Applying a voting process to the user and image recognition process results set allows more accurate analysis of an image (e.g., by decreasing the number of false positives and false negatives). In embodiments, after applying the voting process to create a final result set, the final result set may be displayed to the user.

In embodiments, a user may be a technician using computer aided detection (“CAD”) software to identify areas of interest on an image. In such embodiments, the user may identify areas of interest and input the areas of interest to the CAD software or system. In embodiments, an area of interest may be an area of an image that may represent an item or characteristic that a technician or image recognition process is attempting to identify. For example, in satellite imagery, an area of interest may be an area of a satellite photograph that represents a building, a bunker, or other structure. As another example, in medical imaging, an area of interest may be an area of a medical image that represents cancer, a lesion, a calcification, a tumor, a mass, a fracture, or any other type of ailment, and the user may be a radiologist or other medical doctor. In embodiments, an image may be a photograph, a satellite image, a mammogram, and X-ray image, a gamma ray image, a nuclear image, an MRI image, a computer axial tomography (“CAT”) scan, or any other type of image. Although specific examples are provided, one of skill in the art will appreciate that the systems and methods disclosed herein may be used on any type of image to identify any type of area of interest.

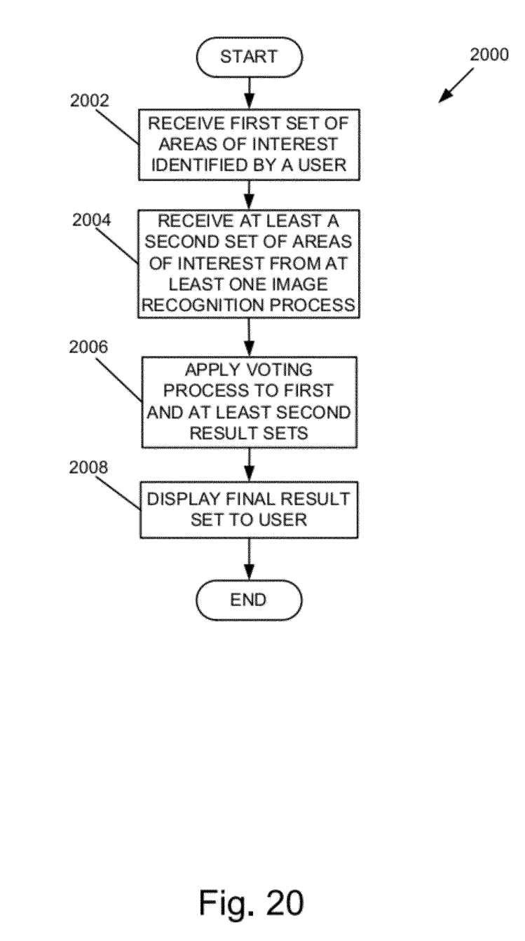

Referring now to FIG. 20, an embodiment of a method 2000 for displaying images using a CAD system is illustrated. In embodiments, the method 2000 may be performed by a general computing device and/or a CAD system. In embodiments, a CAD system may include components of a general computing device as illustrated in FIG. 9. In embodiments, a user interacts with a CAD system to identify areas of interest in an image. For example, the CAD system may display an image to the user. The user may interact with the CAD system to identify areas of interest. For example, by using a touch screen or a peripheral device (e.g., a mouse, keyboard, motion-detection camera, etc.) to input user identified areas of interest into the CAD system. In one embodiment, the user may identify a specific point on the image, a region on the image (e.g., by inputting a border), or may identify the image as a whole as being a single area of interest. A user may input any number of areas of interest (including no areas of interest). In embodiments, the user inputted areas of interest create a first set of areas of interest. At operation 2002, the CAD system receives a first set of areas of interest identified by the user. In embodiments, the CAD system may also store the first set of areas of interest upon receiving it at operation 2002.

In addition to identifying areas of interest, a user may also provide additional information related to the areas of interest to the CAD system. For example, when the user identifies an area of interest, the user may be prompted by the CAD system to enter information about the identified area of interest. Such information may include a user supplied confidence value that identifies the user's confidence that the area of interest is an actual area of interest (e.g., not a false positive or negative), type information (e.g., identifying the area of interest as cancer, a lesions, a calcification, etc.) or any other type of information about the area of interest. In embodiments, the confidence value may be a numerical indicator representing the user's certainty that the user identified area of interest is indeed an actual area of interest. In such embodiments, information is related to the area of interest and received by the CAD system at operation 2002.

In embodiments, one or more image recognition processes are applied to an image to locate areas of interest on the image. In embodiments, each image recognition process may be unique (e.g. each process uses a different algorithm, has a different threshold values, etc.) In embodiments, each image recognition process may identify one or more areas of interest on the image. An image recognition process may also calculate a confidence value for each area of interest that corresponds to the likelihood that an image recognition process properly identified an area of interest.

Flow continues to operation 2004 where the CAD system receives at least a second set of areas of interest from at least one image recognition process. In embodiments, any number of image recognition processes may operate on the image. In cases where multiple image recognition processes are used, a set of identified areas of interest may be received by the CAD system from each image recognition process at operation 2004. In embodiments, the image recognition processes may be a part of the CAD system. In other embodiments, the image recognition processes may not be part of the CAD system, but may communicate with the CAD system over a network or using other means.

Although the method 2000 as illustrated shows receiving a set of areas of interest defined by the user before receiving the one or more sets defined by one or more image recognition processes, one of skill in the art will appreciate that the sets may be received in the opposite order. The method 2000 will operate regardless of the order it receives the sets of areas of interest.

After receiving areas of interest identified by a user and by one or more image recognition process, flow continues to operation 2006 where a voting process is applied to the received sets. In embodiments, the voting process is used to compare the areas of interest received from the user and the one or more image recognition processes. The voting process may calculate a refined confidence value that corresponds to the likelihood that an area of interest is properly identified (e.g., by the user and/or one or more of the image recognition processes). In further embodiments, the voting process may select specific identified areas of interest identified by the user or the one or more image recognition processes, identify new areas of interest based upon the identified areas of interest identified by the user and/or by the one or more image recognition processes, or both. In embodiments, the voting process is any type of function, method, or process that may be employed to refine the results produced by a user and/or one or more image recognition processes in order to create a final result set that more accurately identifies areas of interest. Including the user identified areas of interest in a voting process with the image recognition process(es)' areas of interest allows for the more accurate identification of actual areas of interest. In embodiments, one of the various voting processes described herein may be applied at operation 2006. However, one of skill in the art will appreciate that any type of voting process may be applied at operation 2006. Furthermore, multiple voting processes may also be applied at operation 2006. In embodiments, the voting processes can include the processes described with respect to FIGS. 1-19. In embodiments, multiple image recognition processes may operate upon the image. In such embodiments, a voting process may be performed on the results generated by the multiple image processes to produce a single set of areas of interest. A voting process may then be performed on the single set and the user identified set of voting processes to produce a final set of areas of interest. As such, multiple voting processes may be at operation 2006.

In embodiments, the voting process may produce a final result set containing areas of interest based on the sets received from the user and the one or more image recognition processes. In embodiments, the voting process may use confidence values associated with each received area of interest to determine which areas of interest are most likely actual areas of interest (e.g., not false positives). In embodiments, the voting process may compare the different result sets to each other, the overall characteristics of the image (e.g., the image brightness, contrast, etc.) or to other characteristics in order to adjust the confidence values associated with each area of interest. In embodiments, the final result set may contain only areas of interest whose adjusted confidence value is greater than a predetermined threshold.

In other embodiments, the voting process may produce a final result set of areas of interest based on factors other than confidence values. For example, the voting process may be designed to include the first set of (user provided) areas of interest in the final result set. In such embodiments, it may be preferable to always include areas of interest identified by a user. For example, in situations involving medical imaging, it may be preferable to maintain any areas of interest that a doctor or radiologist identified as suspicious. In such situations, the voting process may ensure that all user identified areas of interest are included in the final set of areas of interest produced by the voting process. In embodiments, in addition to the user identified areas of interest, the final set may also include areas of interest identified by one or more image recognition processes. In this manner, the voting process is used only to provide additional potential positive identifications of areas of interest, but would not eliminate areas that a skilled user might have otherwise identified. For instance, when using a CAD system in mammography, such an embodiment of the voting process guarantees that CAD would never push a radiologist toward making more false negative errors, which mean missing cancer, and may only increase the number of found cancers.

After applying the voting process, flow continues to operation 2008 where the final set of areas of interest are displayed to the user. In one embodiment, displaying the areas of interest to the user may include displaying a copy of the image with one or more indicators identifying areas of interest. In further embodiments, the indicators may differ depending on whether the area of interest was identified by the user or by an image processing algorithm. In further embodiments, displaying the final set of areas of interest may also include displaying information about the areas of interest. In embodiments, information about an area of interest may include a confidence value, type information, or any other information about the area of interest. In embodiments, such information may be provided by the user, by an image recognition process, by a voting process, or by a combination of different sources.

In embodiments, a voting process may be improved by tracking and taking into account the history of the user of the CAD system. For example, a user may be skilled at identifying a certain type of area of interest but have difficulty in correctly identifying a different type of area of interest. For example, a particular radiologist may exhibit a high degree of accuracy when identifying tumors in a mammogram image, but may often fail to identify calcifications. If the voting process is aware of the user's characteristics, the voting process may tailor the final result sets to compensate for the particular user's weakness or may weight more heavily areas of interest that are identified by the user with a strength for identifying such areas.

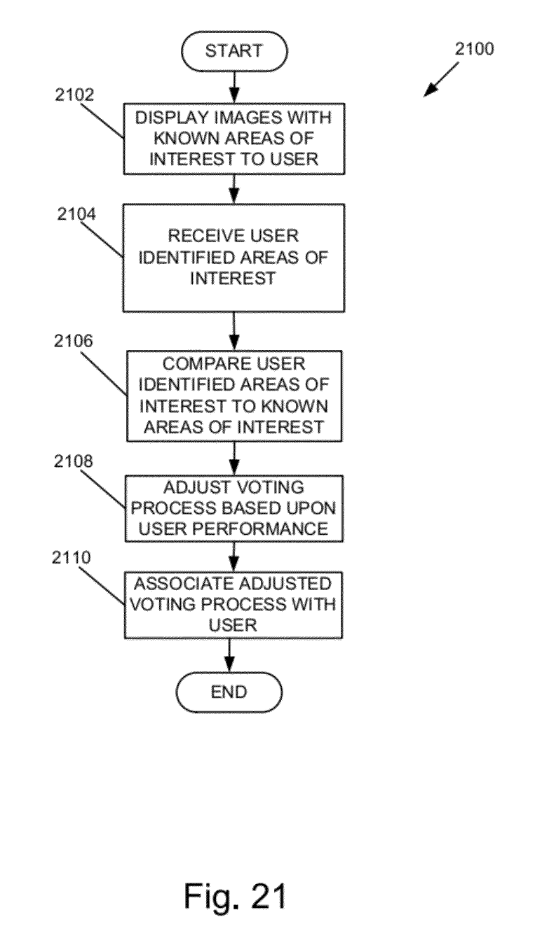

Turning now to FIG. 21, a method 2100 for training a voting process is illustrated. Flow begins at operation 2102 where a number of images with known areas of actual interest are displayed to a user. The images with known areas of actual interest may be images that have been previously analyzed and confirmed to contain a sought after item or characteristic. For example, the known images may be mammogram images that are known to contain cancer, lesions, or be healthy. In embodiments, the user is able to interact with the displayed images and identify one or more areas of interest. As with all forms of practice, the more images that are displayed to the user at operation 2102, the more information will be available to the training method 2100 thereby further improving the voting process.

Flow continues to operation 2104 where the areas of interest indicated by the user on the known images are received by the method 2100. In embodiments, the user indicated areas of interest may be received by a computer performing the training algorithm via a user interface. For example, a user may interact with the display using a touch screen, mouse, keyboard, motion-detection camera, or other mechanism for indicating an area of interest on the displayed image. In further embodiments, along with proving an indication of an area of interest, the user may provide additional information about the area of interest (e.g., type info, confidence value, etc.) Such information may also be received at operation 2104.

Upon receiving the user indicated areas of interest, flow continues to operation 2106 where the user's results are compared against the known results for each image. In embodiments, the user's areas of interest may be compared to the actual areas of interest by measuring the proximity of the user's areas to the known areas, comparing the user provided type information to the actual type information, determining if the user indicated an area of interest where no known area of interest existed, determining if the user did not indicate an area of interest when one or more known areas of interest did exist, or by any other metric for evaluating the accuracy of the user's ability to identify areas of interest. In further embodiments, the comparison at step 2106 may be used to further evaluate the user's ability to correctly identify specific types of areas of interest. For example, the user's ability to identify a mass as opposed to a calcification area in a mammogram image may be rated when performing the comparison at operation 2106. While the example provided relates specifically to medical imaging, one of skill in the art will appreciate that similar ratings are possible in other fields of image analysis.

One of skill in the art will appreciate that the comparison of the user provided areas of interest with the known areas of interest may be performed in many different ways. Any manner of comparing the user results to the known results may be employed at operation 2106 as long as the comparison produces a metric upon which the user's performance can be rated. After evaluating the user's performance, flow continues to operation 2108 where the voting process is adjusted according to the user's performance. In embodiments, adjusting the voting process may include modifying a confidence value threshold used by the voting process to determine whether or not to include an area of interest identified by the user and/or an image recognition process in a final set of areas of interest. For example, if the user displays a high level of skill in identifying areas of interest, the voting process may raise the confidence value threshold for including areas of interest identified by recognition processes in a final result set, thereby reducing the number of false positives that may be included in the result set. Conversely, if the user is not as skilled, the voting process may lower the confidence value threshold at operation 2108. Doing so will include more areas of interest in the final result set, thereby increasing the number of areas of interest in the result set and drawing the user's attention to areas he or she may have missed when performing the initial analysis.

In further embodiments, the voting process may be adjusted to compensate for particular weaknesses of a user. For example, if a user is better at identifying a type A area of interest than a type B area of interest, the voting process may lower the confidence value threshold with respect to areas of interest indicative of type B while maintaining or even raising the confidence value threshold for type A areas of interest. In embodiments, the adjusted voting process, or adjusted parameters related to the voting process, may be saved for future use.

In further embodiments, adjusting the voting process may include adjusting the weight given to the user provided results. For example, if the user successfully identifies areas of interest, the voting algorithm may be adjusted to give the user identified areas of interest, or even lack of identified areas of interest, more weight when determining a final set of areas of interest. In such instances, the adjusted voting process may include more of the user's results, and less of the one or more image recognition processes' results, in the final result set. Conversely, if the user does not successfully identify areas of interest, the user's result set may be given less weight. In such situations, the final result set may include more of the areas of interest identified by an image recognition process than the user.

Because there are many different ways to adjust a voting process, operation 2008 may include creating and/or modifying parameters that may be input to the voting process based upon the user's performance. For example, voting-process parameters may include confidence values, operating instructions for the voting process, threshold values for the voting process, performance tuning input, settings modification, or any other type of input or parameter capable of modifying the functioning of the voting process. The voting-process parameters created or modified at operation 2008 may be stored and later input into the voting process when the specific user, for whom the voting-process parameters were created and/or modified, operates an image detection device or system, such as a CAD system.

Flow continues to operation 2110 where the adjusted voting process is associated with the user. In embodiments, the adjustments made in operation 2108 are applicable to a particular user; however, many different users may operate the computer, such as a CAD system, performing the method 2100. As such, the voting process may be adjusted to particularly suit every user of the system. In such embodiments, the adjustments may be related to a particular user by associating the adjustment with a user ID, a user profile, a smart card or other access mechanism for the user, or by any other means of associating particular data with a user. A user of the CAD system performing actual analysis (as opposed to training) would then have a more tailored experience specific to the user by employing a voting process that has been adjusted for that specific user. In embodiments, a single voting process is used, but user-specific inputs to that voting process affect the output of the voting process. This can be accomplished, in embodiments, by associating with a particular user ID voting-process parameters that are derived for the particular user through the training set forth above. When the user logs in to use the CAD system, such voting-process parameters are provided to the voting process such that the voting process is adjusted to compensate for a user's strengths and weaknesses.

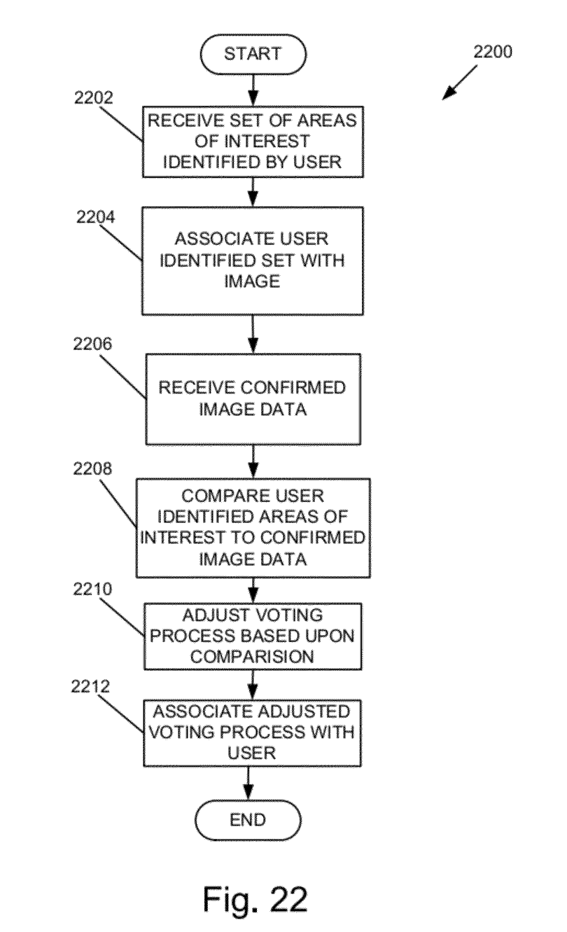

FIG. 22 illustrates another embodiment of a method 2200 for training a voting process. While the method 2100 may be employed to train a user before using the CAD system, the method 2200 may be employed to adjust a voting process during the normal operation of CAD system. Flow begins at operation 2202 where a device or system, such as a CAD system, receives a set of areas of interest for an image identified by a user. In embodiments, the set may contain one or more areas of interest identified by the user. In another embodiment, the set may be empty (e.g., the user indicated that no areas of interest exist on the image). In addition to identifying areas of interest, a user may also provide additional information related to the areas of interest to the CAD system. For example, when the user identifies an area of interest, the user may be prompted by the CAD system to enter information about the identified area of interest. Such information may include a user supplied confidence value that identifies the user's confidence that the area of interest is an actual area of interest (e.g., not a false positive or negative), type information (e.g., identifying the area of interest as cancer, a lesions, a calcification, etc.) or any other type of information about the area of interest. In embodiments, the confidence value may be a numerical indicator representing the user's certainty that the user identified area of interest is indeed an actual area of interest. In such embodiments, information is related to the area of interest and received by the CAD system at operation 2202.

Flow continues to operation 2204 where the set of areas of interest identified by the user are associated with the particular image. In embodiments, associating the set of areas of interest identified by the user with the image may also include storing the set for future use. For example, the association may be accomplished by employing a user ID, a user profile, a smart card or other access mechanism for the user, or by any other means of associating particular data with a user. In embodiments, the set identified by the user may be stored because confirmed image data may not be immediately available. For example, in medical imaging, a doctor may perform a biopsy or other medical procedure on a patient to confirm whether a tumor actually exists. Thus, the user identified set may need to be stored until the confirmed image data is available.

Upon associating and/or storing the user identified set, flow proceeds to operation 2206 where confirmed image data is received by the device or system performing the method 2200. Although not shown, the confirmed data is also associated with an image. For example, a unique identifier may be used to identify a particular image. The user identified set and the confirmed data may be associated with the unique identifier in order to relate the information to the particular image. Flow continues to operation 2208 where the user provided set is compared to the confirmed image data in order to evaluate the user's performance. In embodiments, the user's areas of interest may be compared to the actual areas of interest by measuring the proximity of the user's areas to the known areas, comparing the user provided type information to the actual type information, determining if the user indicated an area of interest where no known area of interest existed, determining if the user did not indicate an area of interest when one or more known areas of interest did exist, or by any other metric for evaluating the accuracy of the user's ability to identify areas of interest. In further embodiments, the comparison at step 2208 may be used to further evaluate the user's ability to correctly identify specific types of areas of interest. For example, the user's ability to identify a mass as opposed to a calcification in a mammogram image may be rated when performing the comparison at operation 2208. While the example provided relates specifically to medical imaging, one of skill in the art will appreciate that similar ratings are possible in other fields of image analysis.

One of skill in the art will appreciate that the comparison of the user provided areas of interest with the known areas of interest may be performed in many different ways. Any manner of comparing the user results to the known results may be employed at operation 2208 as long as the comparison produces a metric upon which the user's performance can be rated. After evaluating the user's performance, flow continues to operation 2210 where the voting process is adjusted according to the user's performance. In embodiments, adjusting the voting process may include modifying a confidence value threshold used by the voting process to determine whether or not to include an area of interest identified by the user and/or an image recognition process in a final set of areas of interest. For example, if the user displays a high level of skill in identifying areas of interest, the voting process may raise the confidence value threshold for including areas of interest in a final result set, thereby reducing the number of false positives that may be included in the result set. Conversely, if the user is not as skilled, the voting process may lower the confidence value threshold at operation 2208. Doing so will include more areas of interest in the final result set, thereby increasing the number of areas of interest in the result set and drawing the user's attention to areas he or she may have missed when performing the initial analysis. In embodiments, this may be accomplished by providing more weight to the user identified areas of interest, by adjusting confidence value thresholds, or by adjusting voting-process parameters as previously described.

Flow continues to operation 2212 where the adjusted voting process is associated with the user. In embodiments, the adjustments made in operation 2210 are applicable to a particular user; however, many different users may operate the computer, such as a CAD system, performing the method 2200. As such, the voting process may be adjusted to particularly suite every user of the system. In such embodiments, the adjustments may be related to a particular user by associating the adjustment with a user ID, a user profile, a smart card or other access mechanism for the user, or by any other means of associating particular data with a user. By associating the adjusted voting process with the user, the voting process can be tailored to compensate for the weaknesses of each particular user of the system. The method 2200 may be performed during normal operation of a CAD system. In doing so, the voting process applied for a particular user may be constantly adjusted to best compliment the user's ability to identify areas of interest as the user's ability develops.

This disclosure describes some embodiments of the present invention with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art.

Although the embodiments have been described in language specific to structural features, methodological acts, and computer-readable media containing such acts, it is to be understood that the possible embodiments, as defined in the appended claims, are not necessarily limited to the specific structure, acts, or media described. One skilled in the art will recognize other embodiments or improvements that are within the scope and spirit of the present invention. Therefore, the specific structure, acts, or media are disclosed only as illustrative embodiments. The invention is defined by the appended claims.

What is claimed is:

1. A method for displaying results using a computer aided detection (CAD) system, the method comprising:
receiving a first set of user identified areas of interest for an image from a user, wherein the first set of user identified areas of interest comprises at least one area of interest identified by the user;
receiving a second set of areas of interest from at least one image recognition process;
applying a voting process to the first set and the second set to produce a result set of areas of interest; and
displaying the final set of areas of interest to the user.

2. The method of claim 1, wherein the first set of user identified areas of interest comprises a confidence value associated with the at least one area of interest identified by the user.

3. The method of claim 2, wherein the final result set comprises all of the user identified areas of interest, regardless of whether the confidence value associated with the at least one area of interest identified by the user is greater than a confidence value threshold set by the voting process.

4. The method of claim 1, wherein the first set of user identified areas of interest comprises type information associated with the at least one are of interest identified by the user.

5. The method of claim 3, wherein the type information comprises information identifying the at least one area of interest as at least one of:
a mass;
a calcification;
a tumor;
a cysts; and
cancer.

6. The method of claim 1, wherein the result set of areas of interest comprise areas of interest with a confidence value greater than a confidence threshold.

7. The method of claim 6, wherein displaying the final set of areas of interest to the user comprises displaying the areas of interest identified by the user in a manner that distinguishes the areas of interest identified by the user from areas of interest identified by the image recognition process.

8. The method of claim 1, wherein the image is one of:
an X-ray image;
a mammography image;
a computer axial tomography scan;
a magnetic resonance imaging;
a nuclear image; and
a gamma ray image.

9. The method of claim 1, wherein the voting process further comprises adjusting the first set and the second set based upon characteristics of the image.

10. The method of claim 1, further comprising:
performing a training process; and
adjusting the voting process based upon the training process.

11. A computer storage medium encoding computer executable instructions that, when executed by at least one processor, perform a method for displaying results using a computer aided detection (CAD) system, the method comprising:
receiving a first set of user identified areas of interest for an image from a user, wherein the first set of user identified areas of interest comprises at least one area of interest identified by the user;
receiving a second set of areas of interest from at least one image recognition process;
applying a voting process to the first set and the second set to produce a result set of areas of interest; and
displaying the final set of areas of interest to the user.

12. The computer storage medium of claim 1, wherein the first set of user identified areas of interest comprises a confidence value associated with the at least one area of interest identified by the user.

13. The method of claim 12, wherein the final result set comprises all of the user identified areas of interest, regardless of whether the confidence value associated with the at least one area of interest identified by the user is greater than a confidence value threshold set by the voting process.
The computer storage medium of claim 1, wherein the first set of user identified areas of interest comprises type information associated with the at least one are of interest identified by the user.

14. The computer storage medium of claim 3, wherein the type information comprises information identifying the at least one area of interest as at least one of:
a mass;
a calcification;
a tumor;
a cysts; and
cancer.

15. The computer storage medium of claim 1, wherein the result set of areas of interest comprise areas of interest with a confidence value greater than a confidence threshold.

16. The computer storage medium of claim 1, wherein the final result set comprises the first set of areas of interest identified by the user and at least one area of interest from the second set of areas of interest.

17. The computer storage medium of claim 6, wherein displaying the final set of areas of interest to the user comprises displaying the areas of interest identified by the user in a manner that distinguishes the areas of interest identified by the user from areas of interest identified by the image recognition process.

18. The method of claim 1, further comprising:
performing a training process; and
adjusting the voting process based upon the training process.

19. A method of training a voting process, the method comprising:
receiving results from a user identifying at least one are of interest on an image;
comparing the results from the user against known results for the image;
based upon the comparison, adjusting at least one voting-process parameter;
storing the voting process parameter; and
providing the voting process parameter for the user to the voting process.

20. The method of claim 19, further comprising adjusting a confidence value threshold based upon the comparison.
